(19) United States
(12) Patent Application Publication
(10) Pub. No.: US 2022/0249479 A1
(43) Pub. Date: Aug. 11, 2022

(54) MODIFIED RELEASE FORMULATION OF A PYRIMIDINYLAMINO-PYRAZOLE COMPOUND, AND METHODS OF TREATMENT

(71) Applicant: DENALI THERAPEUTICS INC., SOUTH SAN FRANCISCO, CA (US)

(72) Inventors: Harish RAVIVARAPU, SOUTH SAN FRANCISCO, CA (US); Travis REMARCHUK, SOUTH SAN FRANCISCO, CA (US); Anantha SUDHAKAR, SOUTH SAN FRANCISCO, CA (US); Bradley K. WONG, SOUTH SAN FRANCISCO, CA (US)

(73) Assignee: DENALI THERAPEUTICS INC., SOUTH SAN FRANCISCO, CA (US)

(21) Appl. No.: 17/614,842

(22) PCT Filed: May 29, 2020
(86) PCT No.: PCT/US2020/035413

§ 371 Date: Nov. 29, 2021

Related U.S. Application Data
(60) Provisional application No. 62/855,740, filed on May 31, 2019.

(51) Int. Cl. A61K 31/506; A61K 9/20; A61K 9/48; A61K 9/50

(52) U.S. Cl. CPC A61K 31/506; A61K 9/2018; A61K 9/2054; A61K 9/2027; A61K 9/2013; A61K 9/5078; A61K 9/4866; A61K 9/5026; A61K 9/5031; A61K 9/501; A61K 9/2009

(57) ABSTRACT

The present disclosure relates to modified release formulations of 2-methyl-2-(3-methyl-4-(4-(methylamino)-5-(trifluoromethyl)pyrimidin-2-ylamino)-IH-pyrazol-1-propanenitrile or solvates, tautomers, and pharmaceutically acceptable salts thereof, and methods of treatment with the modified release formulations.

### CROSS-REFERENCE TO RELATED APPLICATIONS

The present application claims priority to U.S. Provisional Patent Application No. 62/855,740, filed on May 31, 2019, the disclosure of which is incorporated herein by reference in its entirety for all purpose.

### FIELD

The present disclosure relates to formulations of 2-methyl-2-(3-methyl-4-((4- (methylamino)-5-(trifluoromethyl)pyrimidin-2-yl)amino)-1H-pyrazol-1-yl)propanenitrile for use in the treatment of peripheral and neurodegenerative diseases, including Parkinson's disease. The present disclosure also relates to processes to obtain modified release formulations.

### BACKGROUND

Parkinsonism is a term that covers several conditions, including Parkinson's Disease (PD) and other conditions with similar symptoms, collectively known as Parkinsonism, such as slow movement, rigidity (stiffness) and problems with walking. Most people with Parkinsonism have idiopathic Parkinson's disease, also known as Parkinson's. Idiopathic means the cause is unknown. The most common symptoms of idiopathic Parkinson's are tremor, rigidity and slowness of movement. Although the exact causes of Parkinson's disease are unknown, it is believed that a combination of genetic and environmental factors contribute to the etiology of the disease. Drugs approved to treat Parkinson's Disease include dopamine- replacement therapies (levodopa/carbidopa), dopamine agonists (pramipexole, ropinirole, rotigotine, apomorphine), Catechol-O-methyltransferase (COMT) inhibitors (entacapone, levodopa/carbidopa/entacapone, tolcapone, opicapone), Monoamine Oxidase B (MAO-B) Inhibitors (selegiline hydrochloride, rasagiline, safinamide), amantadine, Anticholinergic medications (trihexyphenidyl, benztropine mesylate), Acetylcholinesterase inhibitor (rivastigmine), Serotonin 5-HT_{2A} receptor agonist (pimavanserin), and Dopamine transporter for imaging (ioflupane I-123). However these medications provide symptomatic benefit for Parkinson's disease patients and do not reduce the progression of the disease.

Combined genetic and biochemical evidence implicates certain kinase function in the pathogenesis of neurodegenerative disorders (Christensen, K. V. (2017) Progress in medicinal chemistry 56:37-80; Fuji, R. N. et al (2015) Science Translational Medicine 7(273):273ra15; Taymans, J. M. et al (2016) Current Neuropharmacology 14(3):214-225). Among the genes that been implicated in Parkinson's disease is Park8, which encodes the leucine-rich repeat kinase 2 (LRRK2), a complex signaling protein that is a key therapeutic target, particularly in Parkinson's disease (PD). Mutations in Park8 are found in both familial and non-familial (sporadic) forms of Parkinson's disease, and increased kinase activity of LRRK2 is implicated in the pathogenesis of Parkinson's disease. Mutations in the LRRK2 gene are the most frequent genetic cause of familial Parkinson's disease and a major driver of lysosomal dysfunction, which contribute to the formation of Lewy body protein aggregates and neurodegeneration. LRRK2 regulates lysosomal genesis and function, which is impaired in Parkinson's disease and may be restored by LRRK2 inhibition, thereby potentially reducing disease progression in patients with a genetic LRRK2 mutation as well as in patients with sporadic or idiopathic Parkinson's disease.

LRRK2 kinase inhibitors represent a new class of therapeutics with the potential to address the underlying biology of Parkinson's disease, ALS and other neurodegenerative diseases (Estrada, A. A. et al (2015) Jour. Med. Chem. 58(17): 6733-6746; Estrada, A. A. et al (2013) Jour. Med. Chem. 57:921-936; Chen, H. et al (2012) Jour. Med. Chem. 55:5536-5545; Estrada, A. A. et al (2015) Jour. Med. Chem. 58:6733-6746; Chan, B. K. et al (2013) ACS Med. Chem. Lett. 4:85-90; U.S. Pat. No. 8,354,420; U.S. Pat. No. 8,569,281; U.S. Pat. No. 8,791,130; U.S. Pat. No. 8,796,296; U.S. Pat. No. 8,802,674; U.S. Pat No. 8,809,331; U.S. Pat. No. 8,815,882; U.S. Pat. No. 9,145,402; U.S. Pat. No. 9,212,173; U.S. Pat. No. 9,212,186; US 9,932,325; WO 2011/151360; WO 2012/062783; WO 2013/079493). LRRK2 activity is linked to central mechanisms of Parkinson's disease pathology through its role in lysosomal function. An inhibitor of LRRK2 kinase, a genetically validated target, may improve lysosomal function in LRRK2-PD, and potentially in idiopathic Parkinson's disease. Therefore, LRRK2 inhibition may intervene in an important disease pathway in Parkinson's disease and prevent or curb the accumulation of motor and nonmotor disabilities that define the progression of Parkinson's disease.

There is a need for new therapies aimed to mitigate or delay disease progression and postponement of late motor complications for neurodegenerative disorders. Furthermore, there is a need for solid oral dosage forms of effective pharmaceutical compositions to achieve optimal blood concentrations between the maximum tolerated dose and the minimum effective dose. Optimized solid oral dosage forms modulate release and pharmacokinetic profile, minimize the frequency of dosing, and minimize pill burden in patients with limited swallowing ability and other compliance factors.

### DESCRIPTION

The present disclosure relates to modified release formulations of a pyrimidinylamino- pyrazole kinase inhibitor, referred to herein as the Formula I compound, named as 2-methyl-2- (3-methyl-4-(4-(methylamino)-5-(trifluoromethyppyrimidin-2-ylamino)-1H-pyrazol-1- yl)propanenitrile, and having the structure:

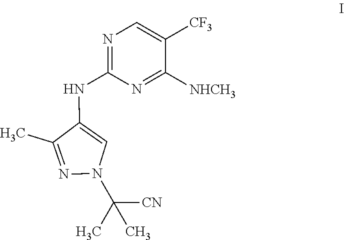 [SMILES: CNc1nc(Nc2cn(C(C)(C)C#N)nc2C)ncc1C(F)(F)F]

or tautomers, polymorphs, or pharmaceutically acceptable salts thereof.

An aspect of the present disclosure includes a modified release formulation comprising a therapeutically effective amount of 2-methyl-2-(3-methyl-4-(4-(methylamino)-5- (trifluoromethyl)pyrimidin-2-ylamino)-1H-pyrazol-1-yl)propanenitrile and at least one release- modifying agent.

An exemplary embodiment of the formulation comprises pellets containing 2-methyl-2- (3-methyl-4-(4-(methylamino)-5-(trifluoromethyppyrimidin-2-ylamino)-1H-pyrazol-1- yl)propanenitrile and coated with the at least one release-modifying agent. In another exemplary embodiment the pellets contain 2-methyl-2-(3-methyl-4-(4-(methylamino)-5- (trifluoromethyl)pyrimidin-2-ylamino)-1H-pyrazol-1-yl)propanenitrile in its core. In another exemplary embodiment the pellets contain an inert core coated with 2-methyl-2-(3-methyl-4-(4- (methylamino)-5-(trifluoromethyl)pyrimidin-2-ylamino)-1H-pyrazol-1-yl)propanenitrile.

An exemplary embodiment of the formulation is wherein release of 2-methyl-2-(3- methyl-4-(4-(methylamino)-5-(trifluoromethyl)pyrimidin-2-ylamino)-1H-pyrazol-1- yl)propanenitrile is less than 60% at two hours and greater than 60% at 8 hours when tested using USP Type-II Apparatus at 50-75 rpm and 37° C. in pH 3 Mcllvaine buffer, wherein the formulation is a tablet.

An exemplary embodiment of the formulation is wherein release of 2-methyl-2-(3- methyl-4-(4-(methylamino)-5-(trifluoromethyl)pyrimidin-2-ylamino)-1H-pyrazol-1- yl)propanenitrile is less than 60% at one hour and greater than 70% at 8 hours when tested using USP Type-II Apparatus at 100 rpm and 37° C. in pH 3 Mcllvaine buffer, wherein the formulation is a capsule containing pellets.

An exemplary embodiment of the formulation is wherein release of 2-methyl-2-(3- methyl-4-(4-(methylamino)-5-(trifluoromethyl)pyrimidin-2-ylamino)-1H-pyrazol-1- yl)propanenitrile is less than 60% at one hour, wherein the formulation is a capsule containing pellets. In some embodiments, less than 60% the compound of Formula I is released in 2 hours (e.g., 5-40% and 5-15%). In some embodiments, less than 60% the compound of Formula I is released in 4 hours (e.g., 15-60% and 15-25%). In some embodiments, less than 60% of the compound of Formula I is released in 12 hours (e.g., 35-55% and 40-60%).

An exemplary embodiment of the formulation is wherein the 2-methyl-2-(3-methyl-4-(4- (methylamino)-5-(trifluoromethyl)pyrimidin-2-ylamino)-1H-pyrazol-1-yl)propanenitrile has a Cₘₐₓ that is decreased relative to an immediate release formulation after administration to a subject (e.g., a human subject).

An exemplary embodiment of the formulation is wherein the Cₘₐₓ is decreased by at least 20% (e.g., 20-80%, 40-80%, 60-80%, and 65-75%).

An exemplary embodiment of the formulation is wherein steady state Cₘₐₓ/Cₘᵢₙ ratio of 2-methyl-2-(3-methyl-4-(4-(methylamino)-5-(trifluoromethyl)pyrimidin-2-ylamino)-1H- pyrazol-1-yl)propanenitrile in blood ranges from about 1.5 to about 4.5 during the first 12 hours after administration to a subject.

An exemplary embodiment of the formulation is wherein the modified release formulation comprises 10% to 50% by weight of 2-methyl-2-(3-methyl-4-(4-(methylamino)-5- (trifluoromethyl)pyrimidin-2-ylamino)-1H-pyrazol-1-yl)propanenitrile.

An exemplary embodiment of the formulation is wherein the 2-methyl-2-(3-methyl-4-(4- (methylamino)-5-(trifluoromethyl)pyrimidin-2-ylamino)-1H-pyrazol-1-yl)propanenitrile is crystalline.

An exemplary embodiment of the formulation is wherein crystalline 2-methyl-2-(3- methyl-4-(4-(methylamino)-5-(trifluoromethyl)pyrimidin-2-ylamino)-1H-pyrazol-1- yl)propanenitrile is milled or micronized.

An exemplary embodiment of the formulation is wherein the release-modifying agent comprises from 3% to 60% by weight of the formulation (e.g., 3-10% about 5%, about 7%, or about 9%).

An exemplary embodiment of the formulation is wherein the release-modifying agent is selected from the group consisting of MCC (Microcrystalline cellulose), HPC (Hydroxypropyl cellulose), HPMC (Hydroxypropyl methylcellulose), PEG (Polyethylene glycol glycerides), PVA (Polyvinyl alcohol), PVP (Polyvinylpyrrolidone), CAP (Cellulose acetate phthalate), CMC-Na (Sodium carboxymethyl cellulose), HPMCAS (Hydroxypropyl methylcellulose acetate succinate), HPMCP (Hydroxypropyl methylcellulose phthalate), Poly(methylacrylate-co-methyl methacrylate-co-methacrylic acid), Poly(methacrylic acid-co-ethyl acrylate), Poly(methacrylic acid-co-methyl methacrylate), CA (Cellulose acetate); CAB (Cellulose acetate butyrate); EC (Ethylcellulose), Poly(ethyl acrylate-co-methyl methacrylate), Poly(ethyl acrylate-co-methyl methacrylate-co-trimethylammonium ethyl methacrylate chloride), PVAc (Polyvinyl acetate), and HPMC/CMC.

An exemplary embodiment of the formulation is wherein the release-modifying agent is selected from the group consisting of Aquacoat®, Walocel®, HP 50/HP 55, Aqoat®, EUDRAGIT® FS 30 D, EUDRAGIT® L 30 D-55/L 100-55, EUDRAGIT® L 12,5/EUDRAGIT® L 100, EUDRAGIT® S 12,5/EUDRAGIT® S 100, Carbopol® polymers, Eastman CA, Eastman CAB, Eastman CAB, Ethocel™, Aquacoat® ECD, or Surelease®, or Glyceride GatteCoat™, EUDRAGIT® NE 30 D, EUDRAGIT® NM 30 D, EUDRAGIT® RL 30 D, EUDRAGIT® RL 100/RL PO, EUDRAGIT® RS 30 D, EUDRAGIT® RS 100/RS, Kollicoat® SR 30 D, Kollidon®, Walocel® HM-PPA, Kollicoat® MAE 30 DP/100 P, and Eastacryl 30 D.

An exemplary embodiment of the formulation is wherein the release-modifying agent is selected from the group consisting of microcrystalline cellulose, hydroxypropyl methylcellulose, polyethylene glycol, polyvinyl alcohol, polyvinyl acetate, polyvinylpyrrolidone, KOLLICOAT®, CARBOPOL®, and AQUACOAT.

An exemplary embodiment of the formulation is wherein the release-modifying agent is polyvinyl acetate.

An exemplary embodiment the release-modifying agent is a mixture of polyvinyl acetate, polyvinylpyrrolidone, and sodium lauryl sulfate. In some embodiments, the mixture of polyvinyl acetate, polyvinylpyrrolidone, and sodium lauryl sulfate is present in about a 90:9:1 ratio. In some embodiments the mixture provides about a 5-9% weight gain coating to a pellet containing 2-methyl-2-(3-methyl-4-(4-(methylamino)-5-(trifluoromethyl)pyrimidin-2-ylamino)- 1H-pyrazol-1-yl)propanenitrile. In some embodiments the mixture provides about a 5% weight gain coating the pellet. In some embodiments the mixture provides about a 6% weight gain coating the pellet. In some embodiments the mixture provides about a 7% weight gain coating to the pellet. In some embodiments the mixture provides about an 8% weight gain coating to the pellet. In some embodiments the mixture provides about a 9% weight gain coating to the pellet.

In an exemplary embodiment of the formulation, the release-modifying agent is KOLLICOAT® SR 30D. In an exemplary embodiment of the formulation, the KOLLICOAT® SR 30D provides about a 5-9% weight gain coating to the pellet. In an exemplary embodiment of the formulation, the KOLLICOAT® SR 30D provides about a 5% weight gain coating to the pellet. In an exemplary embodiment of the formulation, the KOLLICOAT® SR 30D provides about a 6% weight gain coating to the pellet. In an exemplary embodiment of the formulation, the KOLLICOAT® SR 30D provides about a 7% weight gain coating to the pellet. In an exemplary embodiment of the formulation, the KOLLICOAT® SR 30D provides about an 8% weight gain coating to the pellet. In an exemplary embodiment of the formulation, the KOLLICOAT® SR 30D provides about a 9% weight gain coating to the pellet.

An exemplary embodiment of the formulation comprises one or more excipients selected from the group consisting of microcrystalline cellulose, hydroxypropyl methylcellulose, croscarmellose sodium, polyethylene glycol, polyvinyl alcohol, polyvinyl acetate, polyvinylpyrrolidone, purified talc, colloidal silicon dioxide, and magnesium stearate, and a coating.

An exemplary embodiment of the formulation is wherein the formulation is a tablet.

An exemplary embodiment of the formulation wherein the tablet comprises 10 to 500 mg of 2-methyl-2-(3-methyl-4-(4-(methylamino)-5-(trifluoromethyl)pyrimidin-2-ylamino)-1H- pyrazol-1-yl)propanenitrile.

An exemplary embodiment of the formulation is wherein the tablet comprises 40 to 120 mg of 2-methyl-2-(3-methyl-4-(4-(methylamino)-5-(trifluoromethyl)pyrimidin-2-ylamino)-1H- pyrazol-1-yl)propanenitrile.

An exemplary embodiment of the formulation is wherein the tablet comprises 30 to 80 mg of 2-methyl-2-(3-methyl-4-(4-(methylamino)-5-(trifluoromethyl)pyrimidin-2-ylamino)-1H- pyrazol-1-yl)propanenitrile.

An exemplary embodiment of the formulation is wherein the release-modifying agent is HPMC.

An exemplary embodiment of the formulation is wherein the release-modifying agent is a PARTECK® polymer.

An exemplary embodiment of the formulation is wherein the release-modifying agent comprises 20-30% w/w of the formulation.

An exemplary embodiment of the formulation wherein the formulation is a capsule containing pellets.

An exemplary embodiment of the formulation is wherein the capsule is a multi-unit particulate combination of immediate release pellets and modified release pellets contained in the capsule.

An exemplary embodiment of the formulation is wherein the pellets comprise a release- modifying agent selected from KOLLICOAT®, CARBOPOL®, and AQUACOAT®.

An exemplary embodiment of the formulation is wherein the formulation is a multi-unit particulate combination of immediate release pellets and delayed release pellets contained in a capsule.

An exemplary embodiment of the formulation is wherein the modified release formulation is selected from a delayed-release pellet formulation, a controlled-release pellet formulation, an extended-release pellet formulation, and a pulsatile-release pellet formulation.

An exemplary embodiment of the formulation is wherein the formulation comprises a coating agent, wherein the coating agent is EUDRAGIT®.

An exemplary embodiment of the formulation is wherein the coating agent comprises from 3% to 60% of EUDRAGIT® by weight of the formulation.

An exemplary embodiment of the formulation is wherein the coating agent comprises EUDRAGIT® RS 30 D of up to 20% w/w.

An exemplary embodiment of the formulation is wherein the coating agent comprises EUDRAGIT® NM 30 D of up to 60% w/w.

An aspect of the present disclosure includes a method of preparing a modified release formulation comprising:

(a) coating an inert core selected from the group consisting of sugar, MCC and tartaric acid, with 2-methyl-2-(3-methyl-4-(4-(methylamino)-5-(trifluoromethyl)pyrimidin-2- ylamino)-1H-pyrazol-1-yl)propanenitrile to form an API-core pellet;

(b) coating the API-core pellet with a cosmetic, non-functional seal coating to form a seal-coated pellet; and

(c) coating the seal-coated pellet with a release-modifying agent to form the modified release formulation.

An exemplary embodiment of the method of preparing a modified release formulation is wherein the inert core is selected from a sugar, microcrystalline cellulose (MCC), tartaric acid, polyols, carnauba wax, silicon dioxide, and combinations thereof.

An exemplary embodiment of the method of preparing a modified release formulation is wherein the cosmetic, non-functional seal coating is selected from hydroxypropyl methylcellulose (HPMC), and a mixture of hypromellose and ethylcellulose.

An exemplary embodiment of the method of preparing a modified release formulation is wherein the release-modifying agent is selected from the group consisting of KOLLICOAT®, EUDRAGIT®, hydroxypropyl methylcellulose (HIPMC), and a mixture of hypromellose and ethylcellulose.

An aspect of the present disclosure includes a method of preparing a modified release formulation comprising:

(a) roller compaction of 2-methyl-2-(3-methyl-4-(4-(methylamino)-5- (trifluoromethyl)pyrimidin-2-ylamino)-1H-pyrazol-1-yl)propanenitrile and one or more excipients selected from the group consisting of microcrystalline cellulose, hydroxypropyl methylcellulose, croscarmellose sodium, polyethylene glycol, polyvinyl alcohol, polyvinyl acetate, polyvinylpyrrolidone, purified talc, colloidal silicon dioxide, and magnesium stearate, whereby a pellet is formed; and

(b) polymer coating the pellet with a dispersion of a coating agent selected from KOLLICOAT®, CARBOPOL®, AQUACOAT®, and OPADRY® White.

An exemplary embodiment of the method of preparing a modified release formulation further comprises one or more steps selected from extrusion, spheronization, and compression.

An exemplary embodiment of the method of preparing a modified release formulation further comprises filling a soft or hard capsule shell with the coated pellets.

An aspect of the present disclosure includes a method of preparing a modified release formulation tablet comprising:

(a) blending a dry mixture of 2-methyl-2-(3-methyl-4-(4-(methylamino)-5- (trifluoromethyl)pyrimidin-2-ylamino)-1H-pyrazol-1-yl)propanenitrile, povidone, croscarmellose sodium, silicon dioxide, talc, microcrystalline cellulose, and magnesium stearate;

(b) preparing a dry-granulation of the dry mixture by roller compaction as granules;

(c) milling the granules;

(d) adding croscarmellose sodium, silicon dioxide, talc, and magnesium stearate to the milled granules to form an extra-granular mixture;

(e) compressing the extra-granular mixture into tablets; and

(f) coating the tablets with a coating agent selected from KOLLICOAT®, CARBOPOL®, AQUACOAT®, and EUDRAGIT®.

An aspect of the present disclosure includes a method of treating a LRRK2 mediated disease comprising administering to a subject in need thereof a formulation of the present disclosure.

An exemplary embodiment of the method of treating a LRRK2 mediated disease is wherein one or more of the formulations are administered to the subject once per day, twice per day, or three times per day.

An exemplary embodiment of the method of treating a LRRK2 mediated disease is wherein the formulations are administered to the subject twice per day.

An exemplary embodiment of the method of treating a LRRK2 mediated disease is wherein the LRRK2 mediated disease is a neurodegenerative disease.

An exemplary embodiment of the method of treating a LRRK2 mediated disease is wherein the LRRK2 mediated disease is Parkinson's disease.

In accordance with an aspect of the present invention there is provided a steady and consistent blood level of the modified release formulation of the Formula I compound within a therapeutic range of from about 0.2 μM to about 1.2 μM, over a time period of at least 12 hours. Blood concentration may be measured as mean plasma or serum concentrations from multiple subjects or studies. The blood concentration may be measured at time of administration and at various time points to establish a profile of blood concentration in a subject over time after administration of the modified release formulation of the Formula I compound.

The modified-release method of delivery of the present invention may be accomplished by administering multiple single unit dosage forms of equal or varying concentration of the Formula I compound. Each such unit would be designated to release its contents at varying times over at least a twelve hour time period so as to maintain a Formula I compound blood level within the therapeutic range previously described.

A preferred embodiment of the present invention provides that the patient to be treated ingest at a single point in time a dosage form containing the Formula I compound capable of maintaining the patient's blood concentration at from about 0.2 μM to about 1.2 μM over at least a 12 hour time period. Such a dosage form may consist of one or more units, having the same or varying concentrations of the Formula I compound, designed to release its contents at varying times so as to maintain a Formula I compound blood concentration level within the therapeutic range and for the time period previously described.

One embodiment may comprise one single dosage form which contains multiple units within it, which are capable of releasing their contents at varying times (U.S. Pat. No. 5,326,570). Another embodiment of the single dosage form may also consist of one unit capable of immediately releasing a concentration of the Formula I compound, then modified-releasing the Formula I compound at other time points as necessary to maintain blood levels within the therapeutic range. Another embodiment may be for the dosage form to be in multiple separate units capable of releasing the Formula I compound at varying times, the separate multiple units as described above would all be ingested by the patient to be treated at the same time point. Multiparticulates allow flexibility in modifying the therapeutic dose. Capsules can be filled with different amounts of microparticles or pellets without any additional processing or formulation.

### BRIEF DESCRIPTION OF THE DRAWINGS

FIG. 1 shows idealized blood concentrations of the Formula I compound after dosing in an immediate release (IR) formulation of a minimum efficacious dose, a modified release (MR-I) formulation and a modified release formulation at a reduced dose (MR-II).

FIG. 2 shows the ratio of cerebrospinal fluid (CSF) to plasma concentration of the Formula I compound in healthy (non-PD) young and healthy elderly patients on Day 10 of a regimen at different twice per day (BID) doses of an immediate release capsule formulation of the Formula I compound. The mean CSF to unbound plasma ratio was about 1.0. Data shown is from 25, 80, and 100 mg BID multiple dose cohorts.

FIG. 3 shows a modified release tablet with pore former where the Formula I compound and other excipients comprise the core with coatings including Povidone K30 and polyvinyl acetate.

FIG. 4 shows a modified release, matrix tablet where the Formula I compound and other excipients are formulated in a matrix with polyvinyl pyrrolidone and polyvinyl acetate.

FIG. 5 shows a representation of a pellet for a Multiple Unit Pellet System (MUPS) formulation where the inner core of the pellet is an inert material such as sugar, microcrystalline cellulose (MCC), or tartaric acid, covered with a layer of drug, which is seal-coated. The outer layer is a polymeric coating, such as KOLLICOAT® (about 5-12% weight gained relative to the mass of the material to be coated) or EUDRAGIT® for modified release.

FIG. 6 shows a table of comparative formulas, Batch Nos. 1-3 of matrix modified release (MR), 80 mg tablets using 30, 40, and 50% w/w PARTECK® polymers.

FIG. 7 shows comparative dissolution data of Formula I compound MR tablets of FIG. 6. A modified release effect is observed for each of Batch Nos. 1-3 over a time period of 12 hours. Higher % RSD (relative standard deviation) is observed throughout the release profile of Batches 1 and 2 respectively. The release profile of all three batches is similar irrespective of the amount of PARTECK® SRP 80 used. Batch No. 3 containing 50% w/w PARTECK®SRP 80 exhibits low % RSD as compared to Batch Nos. 1 and 2 containing 30% and 40% PARTECK® SRP 80, respectively.

FIG. 8 shows MR matrix Tablets with 10, 15, and 20% w/w HPMC K-15M (intra- granular with direct compression).

FIG. 9 shows comparative dissolution data of MR tablets of FIG. 8.

FIG. 10 shows MR matrix tablets with PARTECK® SRP80 (extra-granular with direct compression).

FIG. 11 shows comparative dissolution data of MR matrix tablets of FIG. 10.

FIG. 12 shows composition of MR (MUPS) Pellets, 80 mg.

FIG. 13 shows comparative drug release data of batches with different pore former (Povidone) levels.

FIG. 14 shows comparative dissolution profiles of Multi-Unit Pellet System (MUPS) capsules with different MR pellets and IR+MR pellets in pH 3 McIlvaine Buffer 900mL (37° C.) at 50 rpm paddle speed with sinker: Samples: 12.02% w/w MR pellets; 5.2% w/w MR pellets; 8.2% w/w MR pellets; and 40 mg IR pellets+40 mg 12.02% w/w MR pellets.

FIG. 15 shows dose-normalized mean concentration-time profiles for Formulations 1-5 in minipig. Modified release (MR) formulations show lower dose-normalized Cmax and generally slower absorption than Formula I compound in capsule (API, active pharmaceutical ingredient) or IR tablets. Samples: API in Gelatin Capsule (1 mg/kg); (4 mg/kg); PARTECK® 40% MR Tablet (80 mg; 4 mg/kg); PARTECK® 30% MR tablet (80 mg; 4 mg/kg); and EUDRAGIT® RS/RL MUPS capsule (1 mg/kg).

FIG. 16 shows dose-normalized data summary for Formulations 1-5 shown in FIG. 15 in Minipig.

FIG. 17A shows mean oral concentration-time plots for Pellet Formulations 1-5 in Minipig. KOLLICOAT® pellets show slower absorption rate. Enteric coated pellets achieved similar exposure to IR pellets. Samples: 1. Uncoated pellets in capsule (immediate release); 2. KOLLICOAT® 8% pellets in capsule; 4. Enteric coated pellets in capsule; and 5. KOLLICOAT® 5% pellets in capsule.

FIG. 17B shows mean concentrations-time plots of Formula I compound in minipigs (N=3) following single oral administration of Formula I compound (1 mg/kg) as uncoated pellets in capsule (immediate release) and MUPS formulations.

FIG. 18 shows Minipig PK modified release Formulations at 1 mg/kg. KOLLICOAT® pellets exhibit slower absorption rate and reduced Cₘₐₓ vs IR pellets. The bioavailability of the KOLLICOAT® 8%: relative to IR was 73%. The bioavailability of the KOLLICOAT® 5%: relative to IR was 86%. Enteric coated pellets achieved similar Cₘₐₓ and AUC (area under the curve) as IR pellets

FIG. 19 shows Cyno PK Modified Release (MR) Formulations at 2 mg/kg.

FIG. 20A shows PK study for Formulations in cynomolgus monkey. Samples: 1. Uncoated pellets in capsule (immediate release); 2. KOLLICOAT® 8% pellets in capsule; 3. API (Formula I compound) in capsule; 4. Enteric coated pellets in capsule; 5. KOLLICOAT® 5% pellets in capsule; 6. KOLLICOAT® 3% pellets in capsule.

FIG. 20B shows mean concentrations-time plots of Formula I compound in monkeys (N=4) following single oral administration of Formula I compound (2 mg/kg) as uncoated pellets in capsule and API in capsule (both immediate release, without polymeric coating) and MUPS formulations.

FIG. 21 shows Modified Release (MR) pellet formulations in capsules with EUDRAGIT® L30D55 and CARBOPOL® applied at the coating stage.

FIG. 22 shows Modified Release (MR) pellet formulations in capsules with AQUACOAT® and CARBOPOL® applied at the coating stage.

FIG. 23 shows Modified Release (MR) pellet formulations in capsules with KOLLICOAT® and CARBOPOL® applied at the coating stage.

FIG. 24 shows compositions of Formula I compound tablets of 40, 80, 100, 106.68 and 160 mg.

FIG. 25 shows manufacturing process steps to prepare Formula I compound tablets of 40, 80, 100, 106.68 and 160 mg.

FIG. 26 shows the mean dissolution profiles of the four modified release tablets with HMPC polymer formulations expressed as percent drug release versus time.

FIG. 27 shows the mean dissolution profiles of the four modified release tablets with HMPC polymer formulations expressed as percent drug release in mg versus time.

FIG. 28 shows the mean dissolution profiles for 40 mg low dose (1A) and 120 mg high dose (2A) tablets with PARTECK® polymer formulations expressed as percent drug release versus time.

FIG. 29 shows the mean dissolution profiles for 40 mg low dose (1A) and 120 mg high dose (2A) tablets with PARTECK® polymer formulations expressed as cumulative drug release versus time.

FIG. 30 shows the mean dissolution profiles from MR pellets with varied polymer coat expressed as drug release vs time in pH 3 Mcllvaine Buffer (900 mL, USP Type-II Apparatus, 100 rpm, 37° C., with sinker).

FIG. 31 shows the mean dissolution profiles for EUDRAGIT®-coated MUPS of Example 4.

### DEFINITIONS

Unless defined otherwise, technical and scientific terms used herein have the same meaning as commonly understood by one of ordinary skill in the art to which this invention belongs, and are consistent with:

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The term “about” or “approximately” in reference to defined parameters, e.g., amounts of an ingredient in a formulation, water content, Cₘₐₓ, tₘₐₓ, AUC, intrinsic dissolution rates, temperature, and time, indicates the inherent variability in, for example, measuring the parameter or achieving the parameter. A person skilled in the art, having the benefit of this disclosure, would understand the variability of a parameter as connoted by the use of the word “about” or “approximately”. When used in conjunction with a numeral, the term “about” or “approximately” includes a +/− (plus or minus) 10% range of that numeral.

“Polymorph”, as used herein, refers to the occurrence of different crystalline forms of a compound differing in packing or conformation/configuration but with the same chemical composition. Crystalline forms have different arrangements and/or conformations of the molecule in the crystal lattice. Therefore, a single compound may give rise to a variety of polymorphic forms where each form has different and distinct physical properties, such as solubility profiles, melting point temperatures, hygroscopicity, particle shape, morphology, density, flowability, compactibility and/or X-ray diffraction peaks. The solubility of each polymorph may vary, thus, identifying the existence of pharmaceutical polymorphs is essential for providing pharmaceuticals with predictable solubility profiles. It is desirable to characterize and investigate all solid state forms of a drug, including all polymorphic forms, and to determine the stability, dissolution and flow properties of each polymorphic form. Polymorphic forms of a compound can be distinguished in a laboratory by X-ray diffractometry and by other methods such as, infrared or Raman or solid-state NMR spectrometry. For a general review of polymorphs and the pharmaceutical applications of polymorphs see G. M. Wall, Pharm Manuf. 3:33 (1986); J. K. Haleblian and W. McCrone, J. Pharm. Sci., (1969) 58:911; “Polymorphism in Pharmaceutical Solids, Second Edition (Drugs and the Pharmaceutical Sciences)”, Harry G. Brittain, Ed. (2011) CRC Press (2009); and J. K. Haleblian, J. Pharm. Sci., 64, 1269 (1975), all of which are incorporated herein by reference.

A “solvate” is a crystal form containing either stoichiometric or nonstoichiometric amounts of a solvent. If the incorporated solvent is water, the solvate is commonly known as a hydrate. Hydrates/solvates may exist as polymorphs for compounds with the same solvent content but different lattice packing or conformation.

The term “hydrate” refers to the complex where the solvent molecule is water.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis -(2- hydroxy-3-naphthoate)) salts. Other salts include acid salts such as coformers described above. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, methanesulfonic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by Stahl P H, Wermuth C G, editors. Handbook of Pharmaceutical Salts; Properties, Selection and Use, 2^{nd} Revision (International Union of Pure and Applied Chemistry). 2012, New York: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

The term “therapeutically effective amount” is an amount of a drug that is low enough to be non-toxic, yet sufficient to achieve a therapeutic result, including eliminating, reducing, and/or slowing the progression of a condition or symptom thereof. The therapeutically effective amount may depend on biological factors. Achieving a therapeutic result can be measured by a physician or other qualified medical personnel using objective evaluations known in the art, or it can be measured by individual, subjective patient assessment.

The term “subject” refers to a mammal to whom a pharmaceutical composition is administered. Exemplary subjects include humans, as well as veterinary and laboratory animals such as monkeys, horses, pigs, minipigs, cattle, dogs, cats, rabbits, rats, mice, and aquatic mammals.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

A “solid oral dosage form” refers to a formulation that is ready for administration to a subject via an oral route. Exemplary oral dosage forms include, but are not limited to, tablets, minitablets, capsules, caplets, powders, pellets, beads, granules, and pelletized tablets containing polymer-coated pellets. A dosage form can be a “unit dosage form,” which is intended to deliver one therapeutic dose per administration.

The term “excipient” refers to a substance formulated with an active pharmaceutical ingredient (API) of a therapeutic medication, included for the purpose of long-term stabilization, bulking up solid formulations that contain potent active ingredients in small amounts, or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors. In some formulations, excipients can be a key determinant of dosage form performance, with effects on pharmacodynamics and pharmacokinetics. Types of excipients for oral dosage formulations include antiadherents, binders, coatings, colors, disintegrants, flavors, glidants, lubricants, preservatives, sorbents, sweeteners, and vehicles.

The term “pellet” encompasses any shape of particle, including beads, granules, irregularly shaped particles and/or spherical particles. The granules can be any suitable size, e.g., about 0.1 mm to about 1.0 mm. In one embodiment, pellet size is about 100 μM (micron) to about 1200 μM (micron), about 100 μM to about 1100 μM, about 150 μM to about 600 μM, or about 100 μM to about 400 μM as measured by methods well known in the art.

“Spheronization” is a rapid and flexible process where pharmaceutical products are made into small spheres, usually involving wetting a dry mixture comprising the API, filler, spheronization agent, binder superdisintegrant, or other excipients a granulation fluid (e.g., water optionally mixed with an alcohol), granulating the wetted mixture, extruding the resulting granulated mass, spheronizing the extrudate to provide beads, and drying the beads. The flow characteristics of spheres makes them suitable for transportation and movement. Spheres provide the lowest surface area to volume ratio and thus pharmaceutical compounds can be coated with a minimum of coating material.

The term “modified release” means that drug release is different from immediate release, i.e., dosage forms that releases about 60% or more of the drug in vivo within about 2 hours. Drug release may alternatively be measured in vitro by the dissolution of the drug in a dissolution medium according to methods known in the art. Examples of modified release profiles include, but are not limited to, modified release, slow release, delayed release, and pulsatile release.

“Release-modifying agent” is a composition, including a polymeric material which may be a mixture of different polymer backbones, chain lengths, and branching which has the property of modifying the release rate of a drug within a formulation. Release-modifying agents alter the release rate of the drug from the dosage form, such that the release rate of a dosage form with a release-modifying agent is different from the release rate of an otherwise identical dosage form, but without the release-modifying agent, under identical conditions. Examples of release-modifying agents include: MCC (Microcrystalline cellulose), HPC (Hydroxypropyl cellulose), HPMC (Hydroxypropyl methylcellulose), PEG (Polyethylene glycol glycerides), PVA (Polyvinyl alcohol), PVP (Polyvinylpyrrolidone), Carbopol, (a) polymers for enteric coating, including: CAP (Cellulose acetate phthalate) such as AQUACOAT®; CMC-Na (Sodium carboxymethyl cellulose) such as WALOCEL®; HPMCAS (Hydroxypropyl methylcellulose acetate succinate) such as AQOAT®; HPMCP (Hydroxypropyl methylcellulose phthalate) such as HP 50/HP 55; Poly(methylacrylate-co-methyl methacrylate-co-methacrylic acid) such as EUDRAGIT® FS 30 D; Poly(methacrylic acid-co-ethyl acrylate) such as EUDRAGIT® L 30 D-55/L 100-55, or KOLLICOAT® MAE 30 DP/100 P, or Eastacryl 30 D; Poly(methacrylic acid-co-methyl methacrylate) such as EUDRAGIT® L 12,5/EUDRAGIT® L 100, or EUDRAGIT® S 12,5/EUDRAGIT® S 100, etc., (b) polymers for time-controlled release, for example: CA (Cellulose acetate) such as Eastman CA and Eastman CAB; (Cellulose acetate butyrate) such as Eastman CAB; EC (Ethylcellulose) ETHOCEL™, or AQUACOAT® ECD, or SURELEASE® (ready-to-use), or Glyceride GATTECOAT™; Poly(ethyl acrylate-co- methyl methacrylate) such as EUDRAGIT® NE 30 D, or EUDRAGIT® NM 30 D; Poly(ethyl acrylate-co-methyl methacrylate-co-trimethylammoniumethyl methacrylate chloride) such as EUDRAGIT® RL 30 D, EUDRAGIT® RL 100/RL PO, EUDRAGIT® RS 30 D, or EUDRAGIT® RS 100/RS; PVAc (Polyvinyl acetate) such as KOLLICOAT® SR 30 D; HPMC/CMC such as WALOCEL® HM-PPA, etc.

### FORMULA I COMPOUND AND PHARMACEUTICAL COMPOSITIONS

The present disclosure includes polymorphs and amorphous forms of Formula I compound, (CAS Registry Number 1374828-69-9), having the structure:

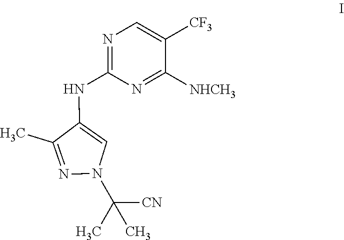 [SMILES: CNc1nc(Nc2cn(C(C)(C)C#N)nc2C)ncc1C(F)(F)F]

and named as: 2-methyl-2-(3-methyl-4-(4-(methylamino)-5-(trifluoromethyl)pyrimidin- 2-ylamino)-1H-pyrazol-1-yl)propanenitrile (WO 2012/062783; U.S. Pat. No. 8,815,882; US 2012/0157427, each of which are incorporated by reference). As used herein, the Formula I compound includes tautomers, or pharmaceutically acceptable salts thereof. The Formula I compound is the API (Active Pharmaceutical Ingredient) in formulations described herein for use in the treatment of Parkinson's Disease and Parkinsonism.

### PHARMACOKINETICS

Dosage form components and configurations can have large effects on the rate of dissolution and blood concentration.

FIG. 1 shows idealized blood concentrations of the Formula I compound after dosing in an immediate release (IR) formulation of a minimum efficacious dose, a modified release (MR) formulation and an MR formulation at a reduced dose.

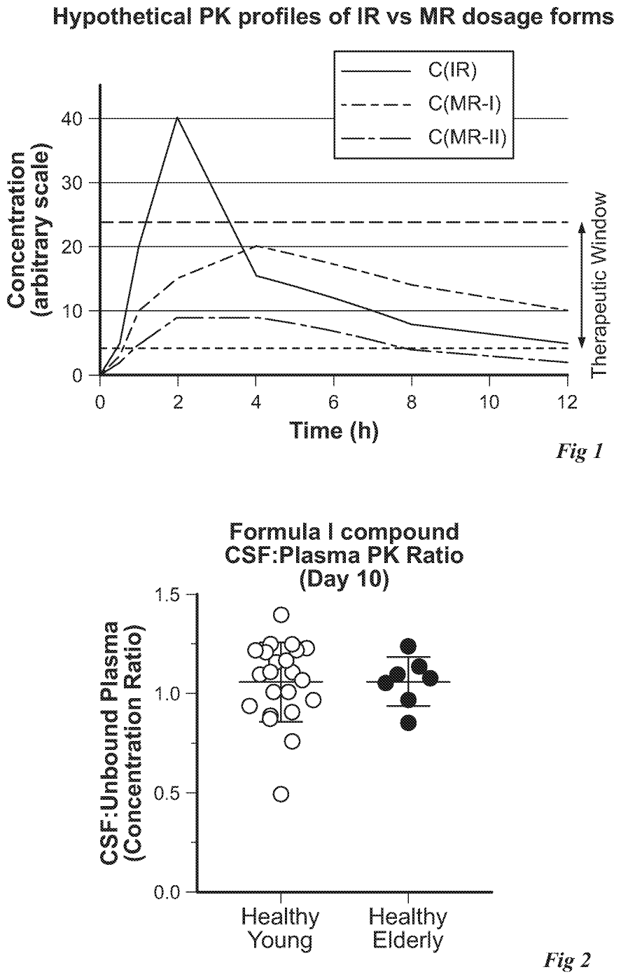

FIG. 2 shows ratio of the cerebrospinal fluid (CSF) to plasma concentration of the Formula I compound in healthy (non-PD) young and healthy elderly patients on Day 10 of a regimen at different twice per day (BID) doses of an immediate release capsule formulation of the Formula I compound. The mean CSF to unbound plasma ratio was about 1.0. Data shown is from 25, 80, and100 mg BID multiple dose cohorts.

Formula I compound was administered to healthy young human subjects as an API-in- capsule formulation at doses of 25 mg, 40 mg, 80 mg and 100 mg BID, and to healthy elder subjects at 80 mg BID. Formula I compound concentrations were determined on Day 1 and 10 and at trough on selected day during the 10 days of administration. Pharmacokinetic analysis of plasma concentrations obtained postdose on Day 10 indicated a terminal half-life in plasma of 14 to 26 hours. A plateau in trough (minimum) concentrations demonstrated that steady-state was achieved by Day 10. Plasma Cmax and AUC increased in a dose-proportional manner over the 25 to 100 mg BID dose range. The terminal half-life, and plasma concentrations and pS935 inhibition at trough (minima) are consistent with twice-daily administration as an efficacious regimen.

With the API in capsule formulation given at doses of 25, 40, 80 and 100 mg BID, the steady-state Formula I compound Cₘₐₓ/Cₘᵢₙ (Cₘₐₓ/C_{trough}) ratio was 2.6 to 12 (mean 5.3). Intrasubject variability in the ratio was seen because of the parallel (non-crossover) nature of the study design. Although generally well tolerated, mild pulse rate and blood pressure changes were noted at the higher doses. Physiologically-based PK modelling, was used to predict the Cₘₐₓ/Cₘᵢₙ ratio for MUPS formulations contain various amounts of KOLLICOAT® polymer. For MUPS formulations containing KOLLICOAT® at 3%, 5% and 8% polymer the predicted Cₘₐₓ/Cₘᵢₙ ratios under BID ranged from 1.5 to 2.6.

### SOLID ORAL DOSAGE FORMS

The present invention provides surprisingly discovered new modified release formulations of the Formula I compound which achieve a desirable modified release profile, and new methodologies for preparing them.

The solid oral dosage forms of the Formula I compound include delivery systems classified broadly into single-unit dosage forms (capsules or tablets) and multiple-unit dosage forms or pelletized dosage forms (pellets or pellets in capsules or tablets). Pellets offer certain therapeutic advantages since they spread uniformly throughout the gastrointestinal tract. Pellets may also empty gradually from the stomach with less intra and inter individual variations, thus giving better predictability for an administered dose. With the use of pellets, the risk of high local drug concentrations and toxicity associated with the intake of locally restricted tablets may be avoided. Premature drug release from enteric-coated tablets in the stomach, potentially resulting in drug degradation or gastric mucosal irritation, can also be reduced with the coated pellets owing to their rapid transit time. The better distribution of pellets in the gastrointestinal tract could also improve the bioavailability of the drug they contain, leading to a possible reduction in drug dose and adverse effects (Kushare, S. et al (2011) Asian J Pharm, 5:203-8).

Immediate Release (IR) dosage forms are formulated to achieve a rapid or uncontrolled release of drug into the patient's blood after administration.

Modified Release (MR), achieves a slower release of drug than that of the conventional, immediate release dosage forms. Advantages of a modified release dosage form include reduced dosing frequency, better patient acceptance and compliance, reduced gastrointestinal (GI) side effects, less fluctuation at plasma drug levels (as measured by the Cₘₐₓ/Cₘᵢₙ ratio), improved efficacy/safety parameters, and a well-characterized and reproducible dosage form. An optimized modified release profile may place the patient in the therapeutic window of more than the minimum effective concentration but less than the maximum tolerated dose of drug for a greater duration post-administration. Modified Release (MR) formulations may achieve a delay in release of the drug into the patient's blood after administration in order to maintain a constant concentration of the drug in the blood.

Multiple Unit Pellet System (MUPS) are multiphasic or programmed release dosage forms, used as an alternative to conventional tablets or capsules. Multiple Unit Pellet System (MUPS) tablets or capsules are a kind of multiparticulate system that has become an important and successful dosage form for immediate or modified drug release for oral administration. These Multiple Units are composed of tablets or capsules containing uncoated or coated pellets allowing modified drug release. Advantages of these systems when compared to simple tablets or capsules include reduction of irritation of the gastric mucosa due to drug degradation of simple units as well as the improvement of dose adjustment. It also offers the possibility to administer incompatible drugs due to the multiparticulate system. Pellets in MUPS tablets or capsules can be uncoated or coated. The drug may be included in the core or as a layer applied to the inert core of the pellet. The inert core may be a neutral starter pellet comprised of sugar, microcrystalline cellulose (MCC), polyols, carnauba wax, or silicon dioxide. In addition, the pellets may have one or more layers that may include suitable excipients for modified release such as polymers for enteric coating or polymers for modified release. Uncoated pellets are made of suitable pharmaceutical excipients such as lactose and microcrystalline cellulose (MCC), among others. The pellets can either be filled into a capsule or compressed into a tablet for oral administration.

Coated pellets are produced with the appropriate polymer and amount to form the coating film. Strength, ductility and thickness properties of the polymer will influence the rupture and deformable capacity of pellets when tableting. In addition, the stability of the pellet's coating film depends on compression forces applied.

Polymers used to create the pellet's coating film include cellulosic and acrylic polymers. Advantages of acrylic polymers are flexibility and features that enable the tableting process without rupture of the pellet's coating film. Combining two types of polymers may improve the coating film flexibility which is desirable for coated pellets elaboration, as well as the addition of a plasticizer in a certain proportion.

The pellet core may influence drug release from MUPS. Pellet porosity of both uncoated and coated pellets affect the modified drug release profile.

The excipients and binder liquid used to produce the pellet core may affect the deformation and viscoelastic properties of the pellet during compression, and thus cause changes in drug release profile. The use of other components like carrageenan polysaccharide in the production/manufacture of the pellets, allows them to have a rapid disintegration and therefore a fast drug release (Kranz H. et al.: Eur. J. Pharm. Biopharm. 73: 302-309 (2009); Ghanam D. and Kleinebudde. P.: Int. J. Pharm. 409: 9-18 (2011).

The manufacturing process of coated pellets can be divided into two steps, pellet manufacture and tablets containing pellets manufacture. First, the drug-pellet manufacturing process begins with the blending of pellet components such as drug, cushioning excipients like microcrystalline cellulose, Glyceryl Monostearate (GMS) and Lactose Monohydrate (LM) which are widely used in this kind of formulation. A binder liquid such as water or glycerol may be used for wet mixing. The mass obtained continues through the extrusion- spheronisation process and the drying of pellets recently formed can be performed in a fluid bed dryer. The next step, pellet coating forms the coating film to obtain the desired drug release (Bashaiwoldu A. B. et al.: Advan. Powder Technol. (2011) 22:340-353).

The tableting process may be performed by a rotary tablet press machine with controlled parameters such as main compression force and speed. Pellets and cushioning excipients may be added for tableting, to optimize certain properties including ability to withstand high compression forces.

Tablets containing pellets with specific features of shape, weight, thickness, and hardness then continue through the tablet film coating process. The tablet film coating is applied to improve the stability and appearance of the pharmaceutical composition.

Film coatings are frequently applied in pharmaceutical drug delivery of solid oral dosage forms. The motivation for coating dosage forms range from cosmetic considerations (color, gloss), improving the stability (light protection, moisture and gas barrier) and making it easier to swallow the tablet. In addition, functional coatings can be used to modify the drug release behavior from the dosage form. Depending on the polymers used it is possible either delay the release of the drug (such as in enteric coatings) or use the coating to sustain the release of the drug from the dosage form over extended periods of time.

A film coating is a thin polymer-based coat applied to a solid dosage form such as a tablet. The thickness of such a coating is usually between 20-100 μm. It is possible to follow the dynamic curing effect on tablet coating structure by using non-destructive analytical methodologies.

Multiple unit pellet systems (MUPS) are designed to obtain a modified release profile of drugs. This modified release may be considered as delayed release or modified release. The delayed release can be achieved, for example, by enteric-coated pellets. Enteric-coating allows that active pharmaceutical ingredients that are unstable in gastric media or may cause gastric irritation are protected by an enteric coating. Methacrylic acid copolymers, hydroxypropylmethylcellulose phthalate, and hydroxypropylmethylcellulose acetate succinate are enteric-coating polymers frequently used for this function.

MUPS tablets containing modified release pellets may achieve sustained action and prolong the pharmacological effect, extend the dosage interval and reduce side effects. Pellets coated with different polymers and different film thicknesses that allow modulation of the release rate from pellets. The polymers used can be, among others, cellulose derivatives such as ethyl cellulose and hydroxypropylmethylcellulose (HPMC). Uncoated pellets may be used as a matrix polymeric system for modified release of the drug. In this group the hydrophilic matrix systems based on the use of cellulosic polymers, carbomers or xanthan gums, among others are frequently used.

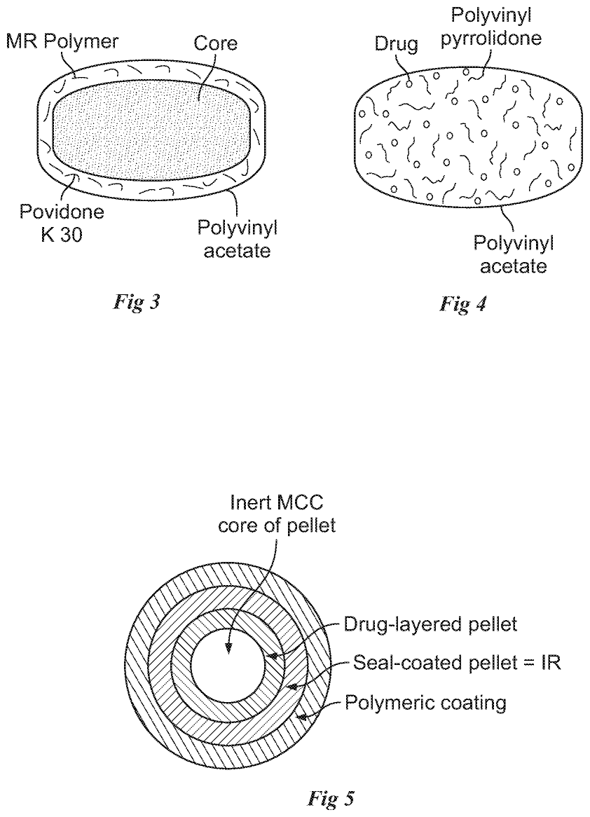

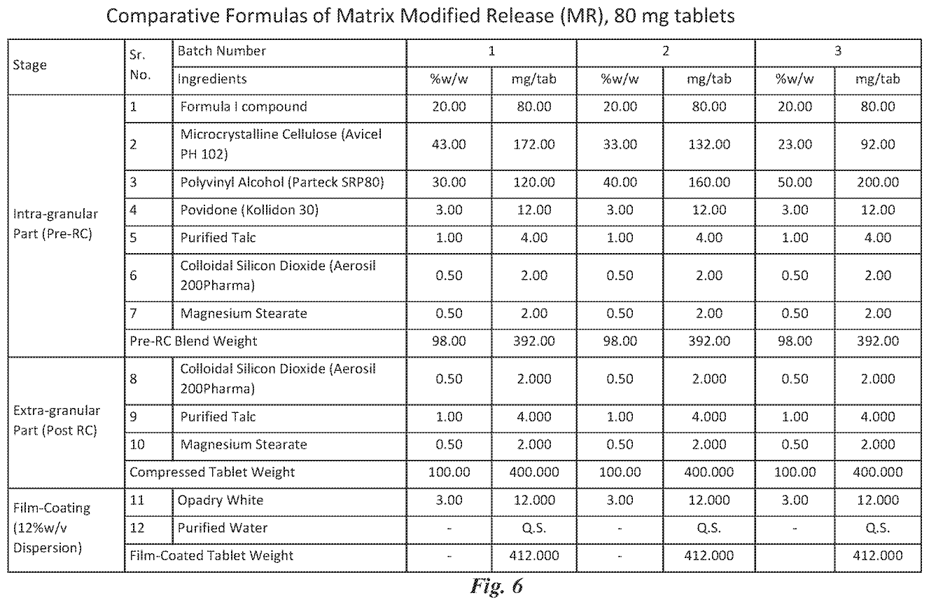

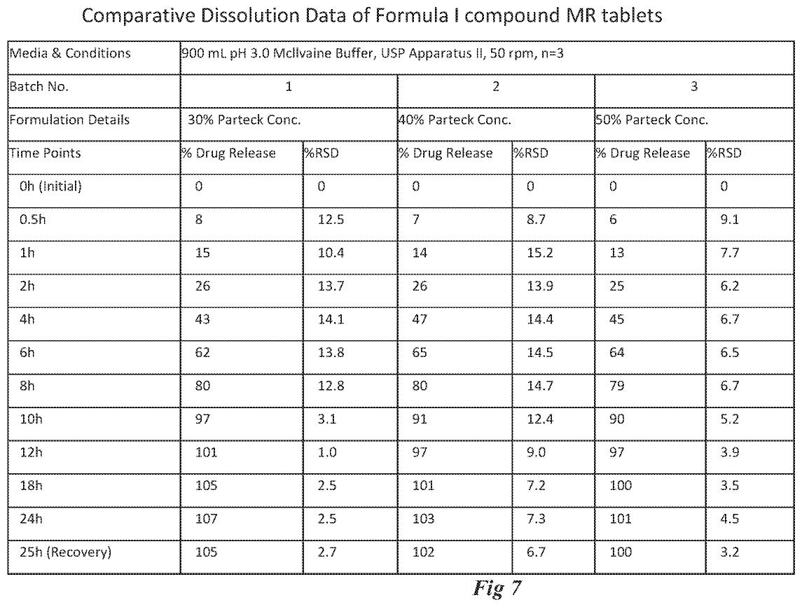

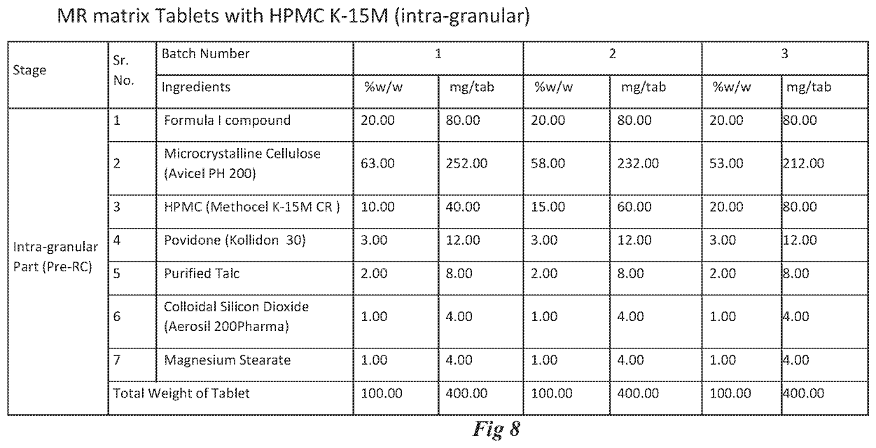

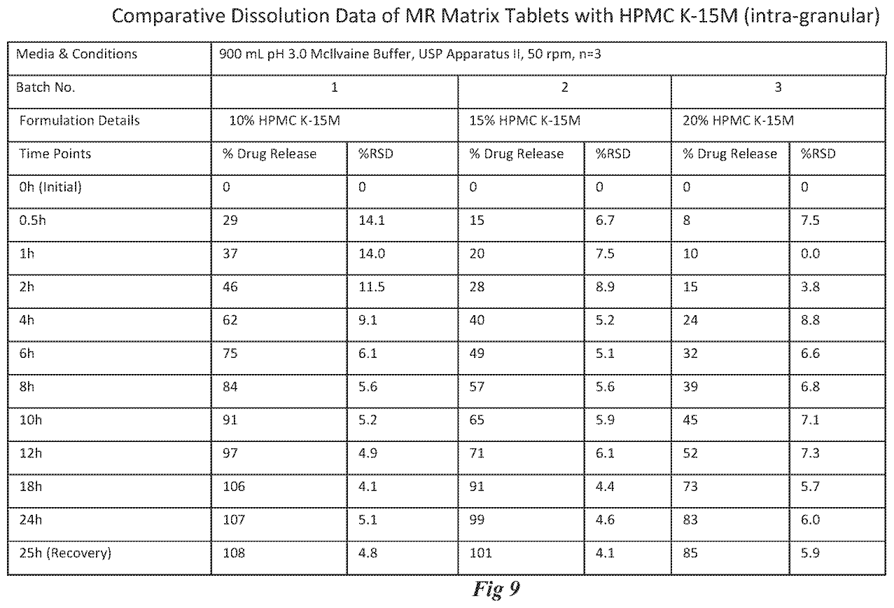

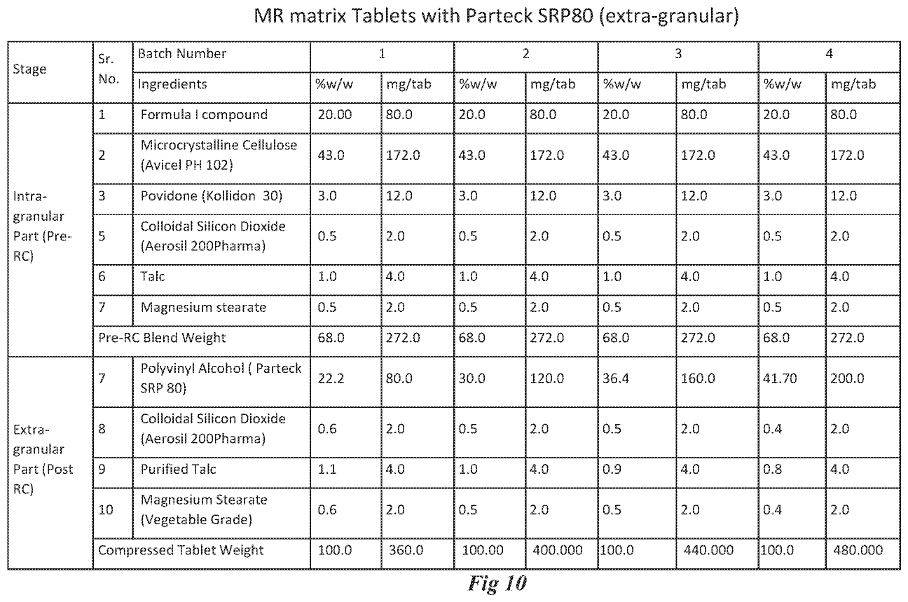

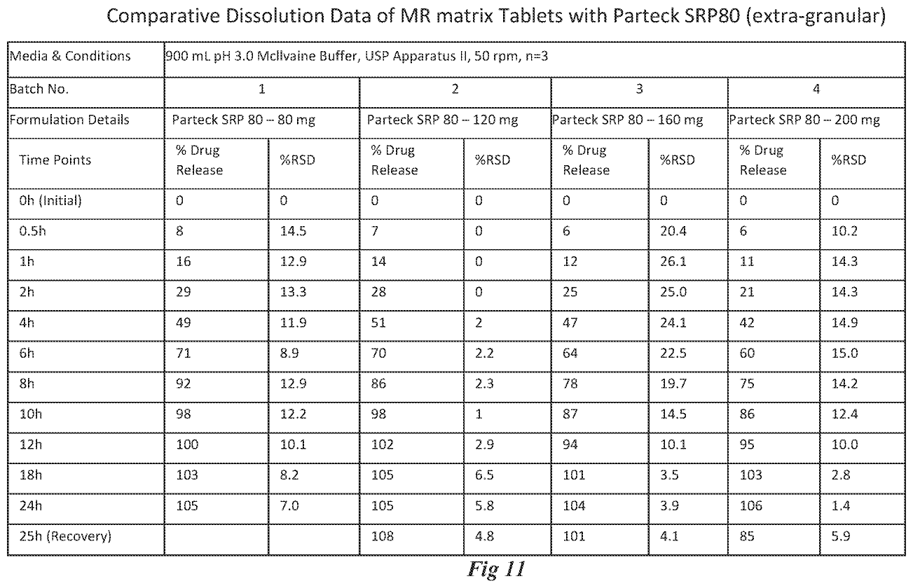

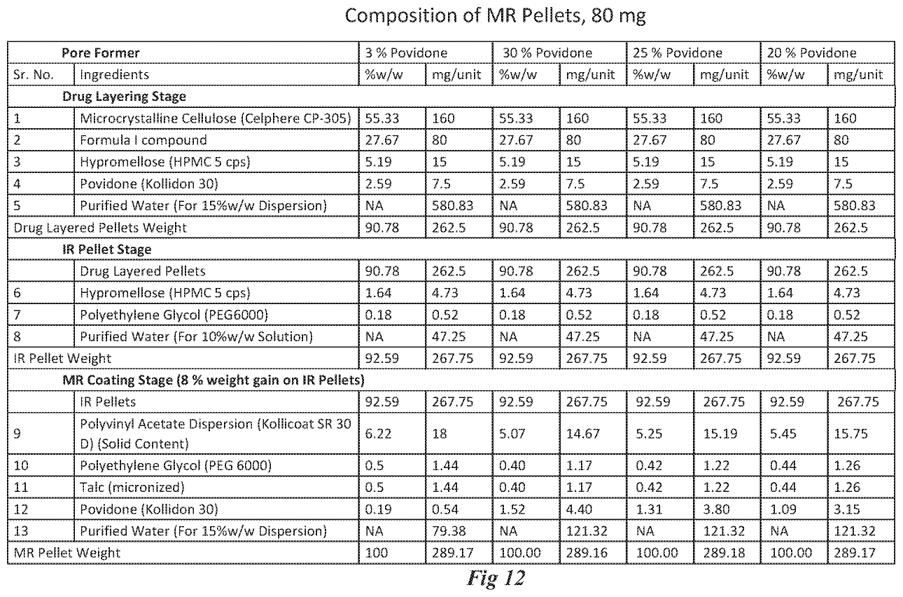

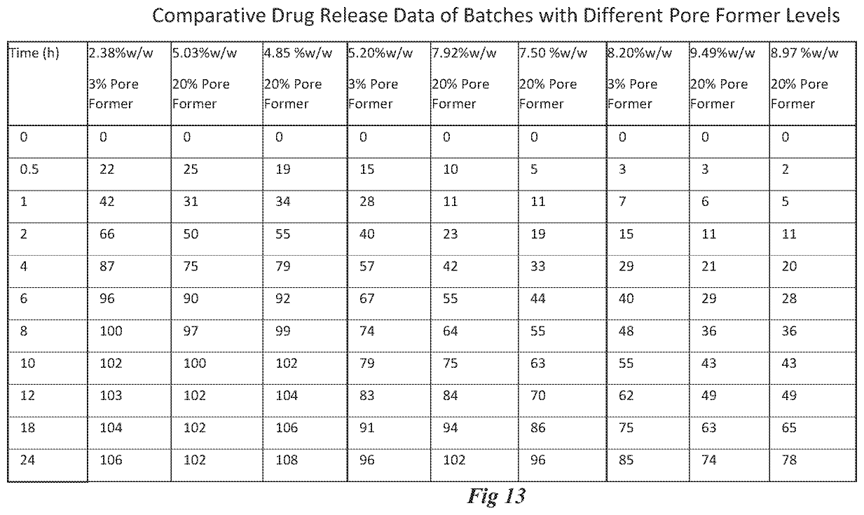

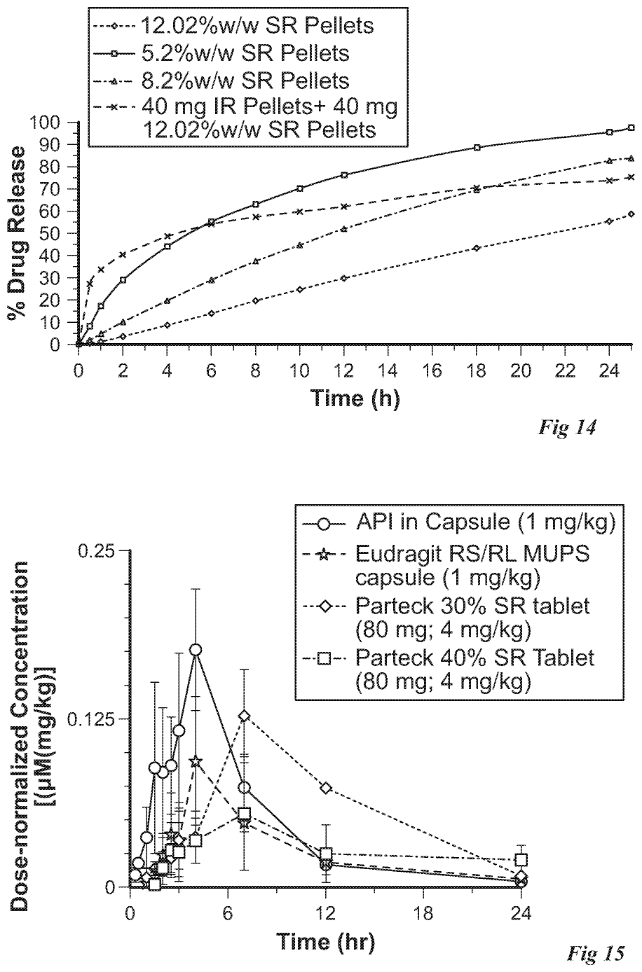

FIG. 3 shows a modified release (MR) tablet with pore former where the Formula I compound and other excipients comprise the core with coatings including KOLLICOAT® IR, Povidone K30 and polyvinyl acetate.

FIG. 4 shows a modified release, matrix tablet where the Formula I compound and other excipients are formulated in a matrix with polyvinyl pyrrolidone and polyvinyl acetate.

FIG. 5 shows a representation of a pellet for a Multiple Unit Pellet System (MUPS) formulation where the inner core of the pellet is an inert material such as sugar, microcrystalline cellulose (MCC), or tartaric acid, covered with a layer of drug, which is seal-coated. The outer layer is a polymeric coating, such as KOLLICOAT® (about 5-12%) for modified release, or EUDRAGIT® for extended release.

### EXCIPIENTS

Suitable excipients are known to those skilled in the art and include materials such as carbohydrates, waxes, water soluble and/or swellable polymers, hydrophilic or hydrophobic materials, gelatin, oils, solvents, water and the like. Excipients can have various and multiple effects and useful properties.

PARTECK® SRP 80 (EMD Millipore) is a functional excipient based on the hydrophilic polymer polyvinyl alcohol (PVA). It forms a swellable and erodible matrix and is used in the formulation of pharmaceutical oral dosage tablet forms showing a modified API release. PARTECK® SRP 80 contains a single ingredient—PVA 40-88—with no further additives, viscosity in mPa of a 4% aqueous solution at 20° C88: degree of hydrolysis (saponification) in mol %. PARTECK® SRP 80 is milled polyvinyl alcohol (PVA 40-88) with a special particle size. CAS Registry Number 9002-89-5

KOLLICOAT® SR 30D (BASF) is an aqueous dispersion of polyvinyl acetate stabilized with Povidone and SLS (sodium lauryl sulfate). KOLLICOAT® SR 30D contains about 27% polyvinyl acetate, about 2.7% povidone K30, about 0.3% sodium lauryl sulphate, and about 70% water (CAS Registry No. 9003-20-7). Polyvinyl acetate, povidone (polyvinylpyrrolidone), and sodium lauryl sulfate are present in about a 90:9:1 ratio. PVA form an insoluble matrix and reduces the drug release. The povidone added in aqueous dispersion is highly soluble in nature and when the tablet comes into contact with the dissolution media, it dissolves and acts as a pore-forming agent. The drug dissolves and diffuses out through the pores at a controlled rate, leaving an empty polymer shell. The viscosity of povidone (PVP K30 Vs PVP K90) and its concentration, both impacts the drug release. As the viscosity and concentration of PVP increases, the drug release increases.

Povidone (polyvinylpyrrolidone, PVP) is a synthetic polymer vehicle used for dispersing and suspending drugs. It also acts as a disintegrant and tablet binder. It appears as a white to off- white hygroscopic powder in its pure form and is readily soluble in water.

Hypromellose, also known as hydroxypropyl methylcellulose and HPMC, is a semisynthetic, inert, viscoelastic, water-soluble polymer used as an excipient and controlled- delivery component in oral medicaments, and found in a variety of commercial products. is used for its seal coating effect such as to create a smooth surface. In other uses HPMC can quickly hydrate on the outer tablet skin to form a gelatinous layer. A rapid formation of a gelatinous layer prevents wetting of the interior and disintegration of the tablet core. Once the original protective gel layer is formed, it controls the penetration of additional water into the tablet. As the outer gel layer fully hydrates and dissolves, a new inner layer replaces it and is cohesive and continuous enough to retard the influx of water and control drug diffusion. A fast rate of hydration followed by quick gelation and polymer/polymer coalescing is necessary for a rate-controlling polymer to form a protective gelatinous layer around the matrix. This prevents the tablet from immediately disintegrating, resulting in premature drug release. The optimized amount of polymer content, such as HPMC, in a matrix system forms a uniform barrier to protect the drug from immediately releasing into the dissolution medium. If the polymer level is too low, a complete gel layer may not form. Increased polymer level in the formulation results in decreased drug-release rates. Because hydrophilic matrix tablets containing HPMC absorb water and swell, the polymer level in the outermost hydrated layers decreases with time. The outermost layer of the matrix eventually becomes diluted to the point where individual chains detach from the matrix and diffuse into the bulk solution. The polymer chains break away from the matrix when the surface concentration passes a critical polymer concentration of macromolecular disentanglement or surface erosion. The polymer concentration at the matrix surface may be defined as the polymer disentanglement concentration.

METHOCEL® (The Dow Chemical Co.) is a commercial line of HPMC products, designated E, F, K, etc., and used frequently for controlled-release drug formulations. The Methocel products differ by viscosity at certain concentrations in water. K15M refers to a high molecular weight HPMC having about 19-24% methoxyl, about 7-12% hydroxypropoxyl, and viscosity (2% in water at 20° C.) of 10,000-18,000 cP (centipoise). K100LV refers to a low molecular weight HPMC having about 19-24% methoxyl, about 7-12% hydroxypropoxyl, and viscosity (2% in water at 20° C.) of 80-120 cP (centipoise).

EUDRAGIT® (Evonik) is a family of proprietary, targeted drug release coating polymethacrylate polymers. EUDRAGIT® polymers can be acidic, neutral or basic and thus be either controlled time release or pH-dependent, and thus either delayed or sustained release as well. These polymers allow drugs to be formulated in enteric, protective or sustained-release formulations to prevent break-down of the drug until it has reached an area with adequate pH in the gastrointestinal (GI) tract. Once the drug reaches its target area of the gastrointestinal tract (i.e., duodenum, stomach) it can release from the polymer matrix and be absorbed. CARBOPOL® (Lubrizol) is a family of high molecular weight, crosslinked polyacrylic acid polymers used as a coating agent. Carbopols form hydrogels in water or alkaline solution due to the hydration of the carboxyl groups, and may be used a release-modifying agent in tablets or pellets formulations.

Sodium croscarmellose, or croscarmmellose sodium, is an internally cross-linked sodium carboxymethylcellulose for use as a disintegrant in pharmaceutical formulations, providing drug dissolution and disintegration characteristics.

AQUACOAT ECD® (FMC Biopolymer) is a 30% (w/w) aqueous dispersion of ethylcellulose (EC) polymer. Ethylcellulose is a hydrophobic coating material used in a variety of coatings applications to achieve sustained release, taste masking and moisture barrier/sealant. AQUACOAT® ECD is a 30% by weight aqueous dispersion of ethylcellulose polymer.

Cushioning agents, such as polyethylene glycol, may be used to prevent pellet deformation during compaction.

A non-functional “Coating agent” such as OPADRY® provides a cosmetic effect, such as color, without modifying the release rate of a drug within a formulation.

### MODIFIED RELEASE FORMULATIONS

The Formula I compound is formulated in accordance with standard pharmaceutical practice and according to procedures of Example 2, for use in therapeutic treatment (including prophylactic treatment) in mammals including humans. The present disclosure provides various formulations comprising the Formula I compound in association with one or more pharmaceutically acceptable excipients. A modified-release drug formulation releases active ingredients over several hours, in order to maintain a constant concentration of the drug in the blood.

The formulations may be prepared using conventional dissolution, blending and mixing procedures. The compound of the present disclosure is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

### PHARMACOKINETICS OF MR FORMULATIONS IN MONKEYS AND MINIPIGS

FIG. 15 shows Dose-Normalized Mean Concentration-Time Profiles for Formulations 1-5 in Minipig. Modified release (MR) formulations show lower dose-normalized Cₘₐₓ and generally slower absorption than Formula I compound in capsule (API) or IR tablets. Samples: API in Gelatin Capsule (1 mg/kg); PARTECK® 40% MR Tablet (80 mg; 4 mg/kg); PARTECK® 30% MR tablet (80 mg; 4 mg/kg); and EUDRAGIT® RS/RL MUPS capsule (1 mg/kg; Example 4).

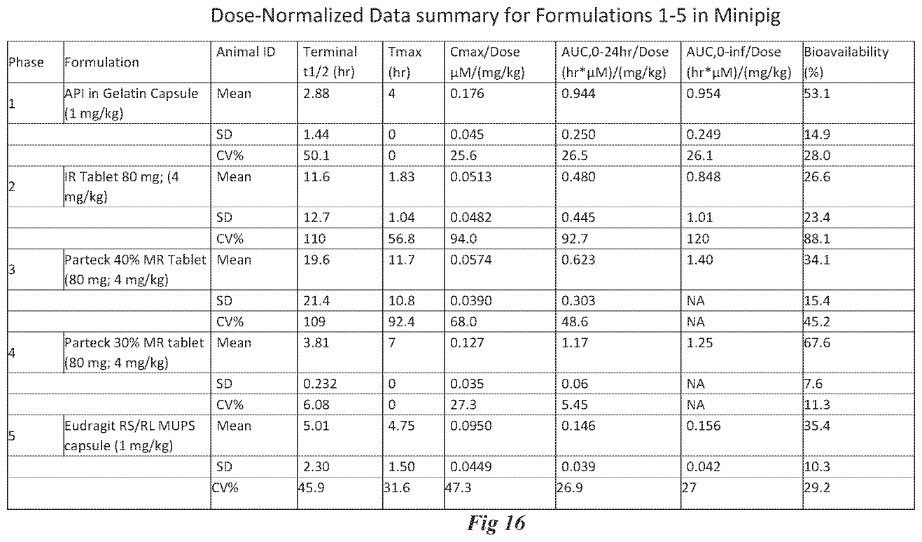

FIG. 16 shows Dose-Normalized Data summary for Formulations 1-5 shown in FIG. 15 in Minipig.

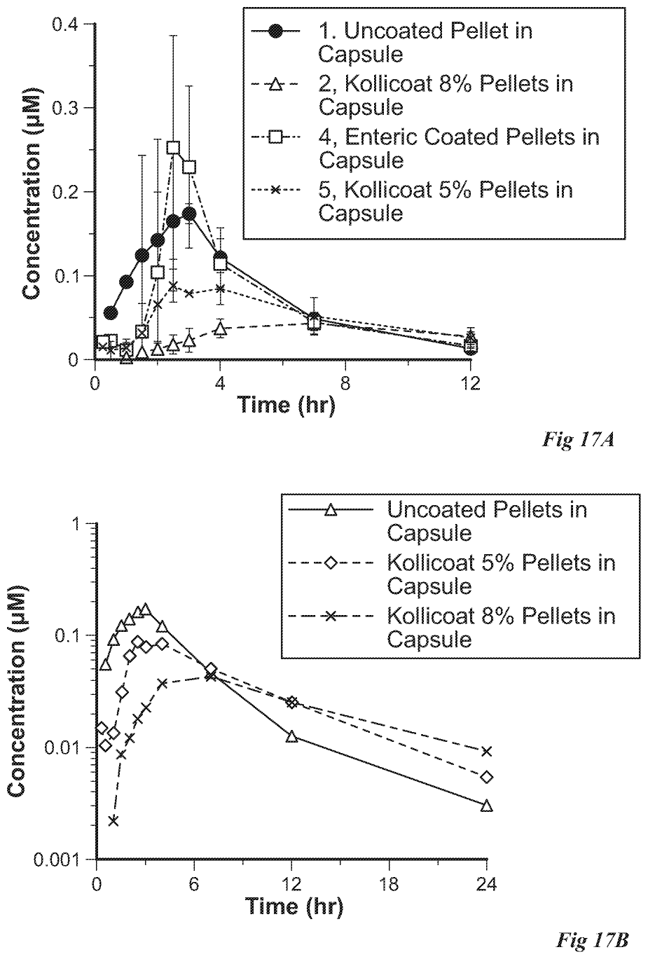

FIG. 17A shows mean oral concentration-time plots for Formulations 1-5 in minipigs. The uncoated drug pellet (IR), and KOLLICOAT® 5% and 8% formulations in capsules were evaluated using a crossover design in fasted Gottingen minipigs (N=3). KOLLICOAT® pellets show slower absorption rate. Enteric coated pellets achieved similar exposure to IR pellets. 1. IR pellets in capsule; 2. KOLLICOAT®8% pellets in capsule; 3. IV (0.5 mg/kg); 4. Enteric coated pellets in capsule; and 5. KOLLICOAT®5% pellets in capsule. The KOLLICOAT® 5% and 8% formulations at 1 mg/kg exhibited slower absorption of Formula I compound. Median Tₘₐₓ values were 2.0, 2.5 and 4.0 hr for the uncoated pellet, and KOLLICOAT® 5% and 8% formulations, respectively, while the corresponding Cₘₐₓ values were 0.197, 0.0940 and 0.0469 respectively.

FIG. 17B shows mean concentrations-time plots of Formula I compound in minipigs (N=3) following single oral administration of Formula I compound (1 mg/kg) as immediate release and MUPS formulations. In contrast to the case in monkeys, the bioavailability of the KOLLICOAT® formulations was similar or slightly lower than the uncoated pellet formulation; the relative bioavailabilities of the KOLLICOAT® 5% and 8% formulations were 86% and 73%, respectively. Overall, the MUPS formulations exhibited slower absorption rate and lower Cₘₐₓ than immediate-release formulations.

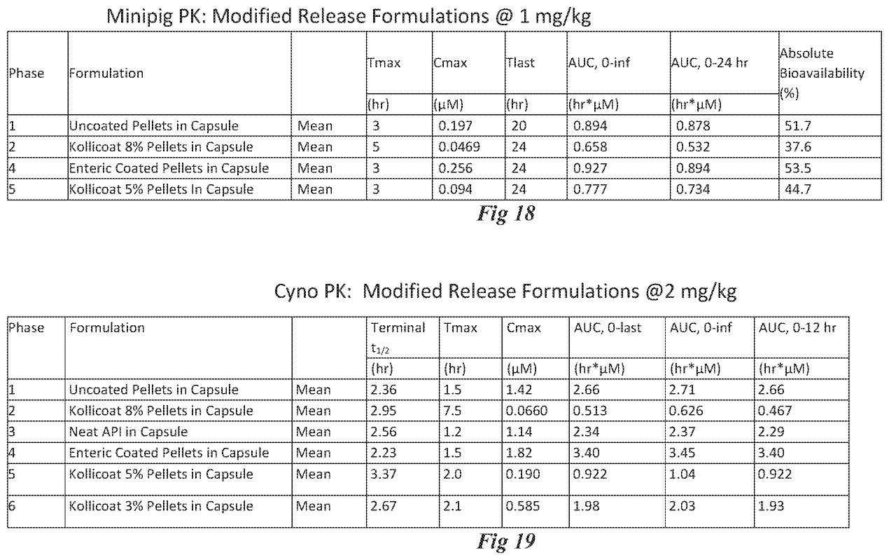

FIG. 18 shows Minipig PK: Modified Release Formulations at 1 mg/kg. KOLLICOAT® pellets exhibit slower absorption rate and reduced Cₘₐₓ vs IR pellets. The bioavailability of the KOLLICOAT® 8%: relative to IR was 73%. The bioavailability of the KOLLICOAT® 5%: relative to IR was 86%. Enteric coated pellets achieved similar Cₘₐₓ and AUC as IR pellets.

FIG. 19 shows Cyno PK: Modified Release (MR) Formulations at 2 mg/kg. KOLLICOAT® pellet formulations exhibited slower rate of absorption. The reduction in bioavailability was relative to immediate release capsule formulations. The magnitude of reduction was dependent on % pellet coating, where a higher coating gave lower F. The enteric coated pellet formulation gave no improvement relative to the immediate release formulation (IR).

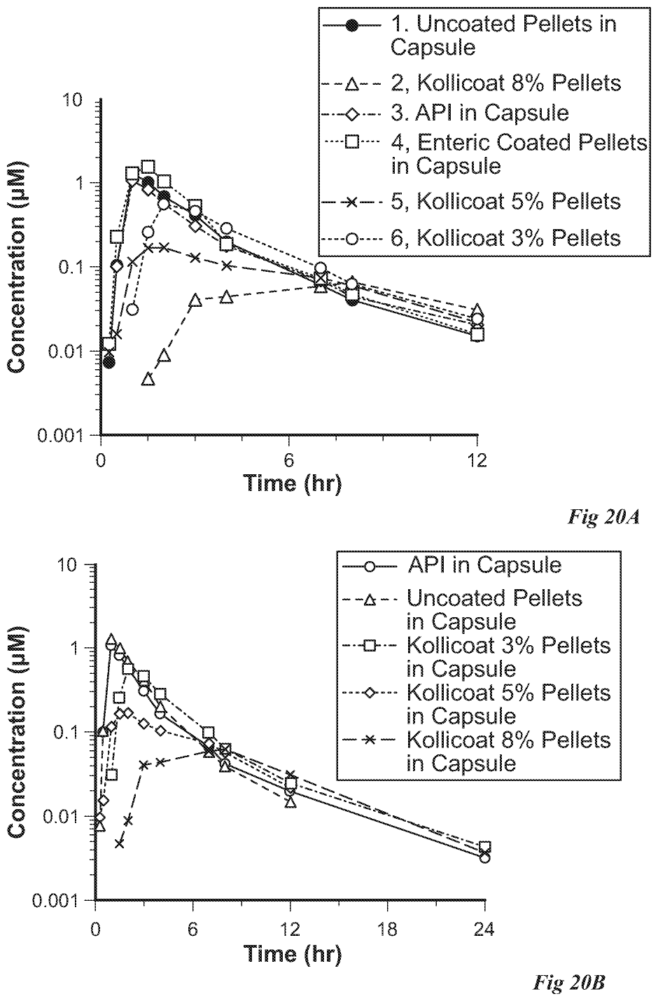

FIG. 20A shows PK study for Formulations in Cynomolgus monkey (body weight approximately 5 kg). 1. IR pellets in capsule; 2. KOLLICOAT® 8% pellets in capsule; 3. API (Formula I compound) in capsule; 4. Enteric coated pellets in capsule; 5. KOLLICOAT® 5% pellets in capsule; 6. KOLLICOAT® 3% pellets in capsule. The MUPS formulations contained the Formula I compound formulated as drug layered pellets coated with KOLLICOAT® SR 30D polymer at various levels (3%, 5% and 8% w/w) designed to provide different drug release rates. In vitro dissolution results supported further characterization with in vivo PK studies. The MUPS formulations were evaluated in cynomolgus monkeys using a cross-over design with a minimum one-week washout period. The uncoated pellet (IR) and API in capsule formulations served as comparators with immediate release rates. A single dose (2 mg/kg Formula I compound) of each formulation was administered to fasted animals (n=4) and timed blood samples were obtained over 24 hr post-dose.

Following oral administration to fasted monkeys, the uncoated pellet and API in capsule formulations achieved similar Tₘₐₓ, Cₘₐₓ and AUC_{0-inf}. The formulations containing KOLLICOAT® SR 30D coated pellets exhibited slower absorption of Formula I compound relative to the two immediate-release formulations as shown by a longer Tₘₐₓ and reduced Cₘₐₓ. FIG. 20B shows mean concentrations-time plots of Formula I compound in monkeys (N=4) following single oral administration of Formula I compound (2 mg/kg) as immediate release and MUPS formulations. The median Tₘₐₓ for the API in capsule formulation was 1.25 hr as compared to 2.0, 1.75 and 7.5 hr for the KOLLICOAT® 3%, 5% and 8% formulations respectively, while the corresponding mean Cₘₐₓ values were 1.14, 0.585, 0.190 and 0.0660 μM respectively. The relative bioavailabilities, based on AUC ratios compared to the API in capsule, of the KOLLICOAT® 3%, 5% and 8% formulations were 84%, 40% and 20%, respectively, which indicated that the lower Cₘₐₓ for the two formulations with higher polymer content was due to a combination of slower absorption rate and a decrease in the extent of absorption.

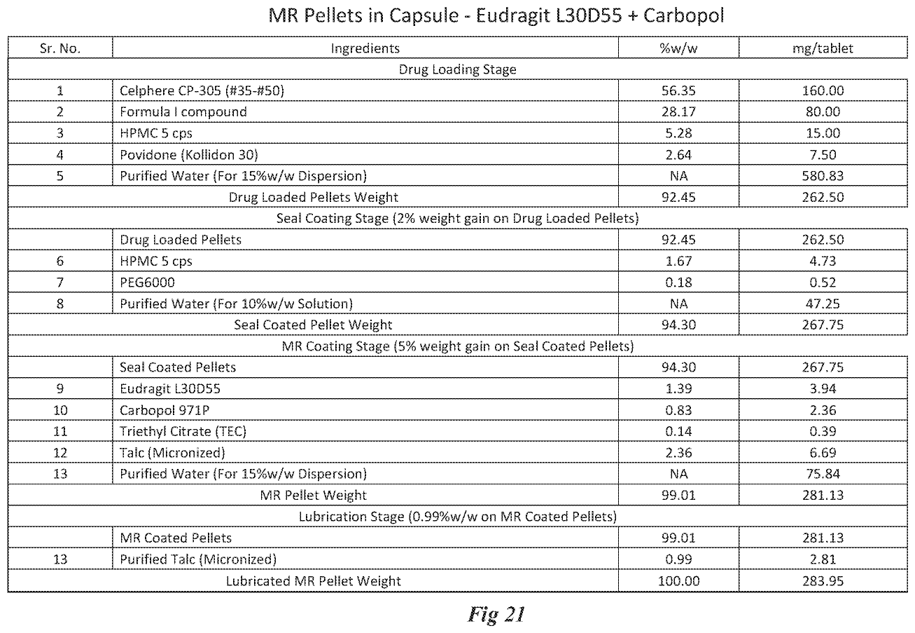

FIG. 21 shows Modified Release (MR) pellet formulations in capsules with EUDRAGIT® L30D55 and CARBOPOL® applied at the coating stage.

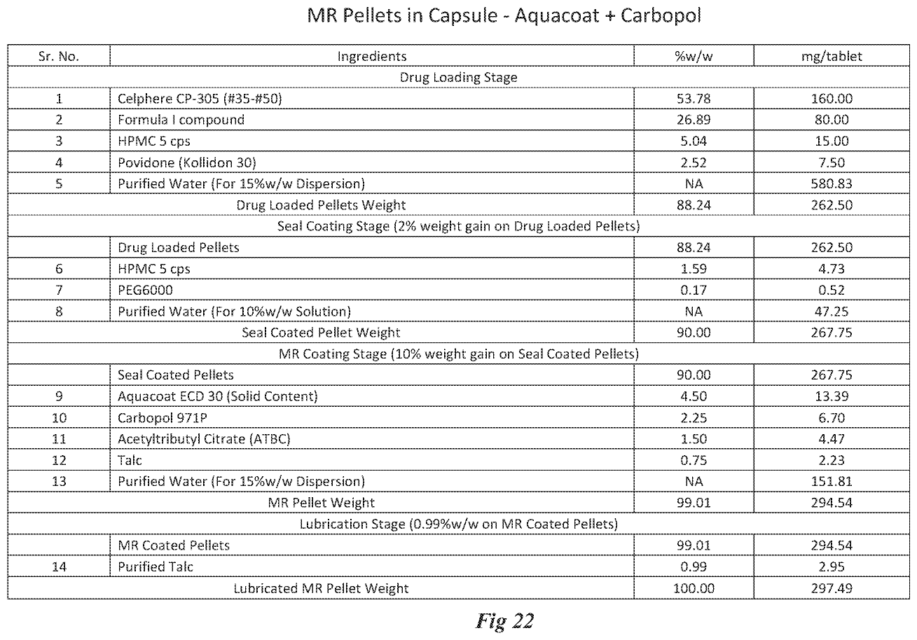

FIG. 22 shows Modified Release (MR) pellet formulations in capsules with AQUACOAT® and CARBOPOL® applied at the coating stage.

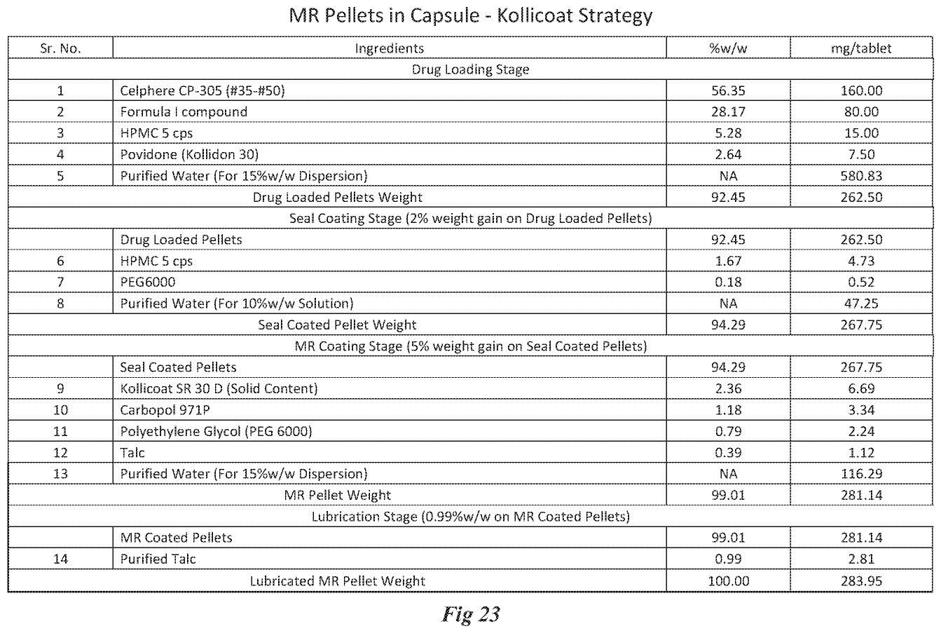

FIG. 23 shows Modified Release (MR) pellet formulations in capsules with KOLLICOAT® and CARBOPOL® applied at the coating stage.

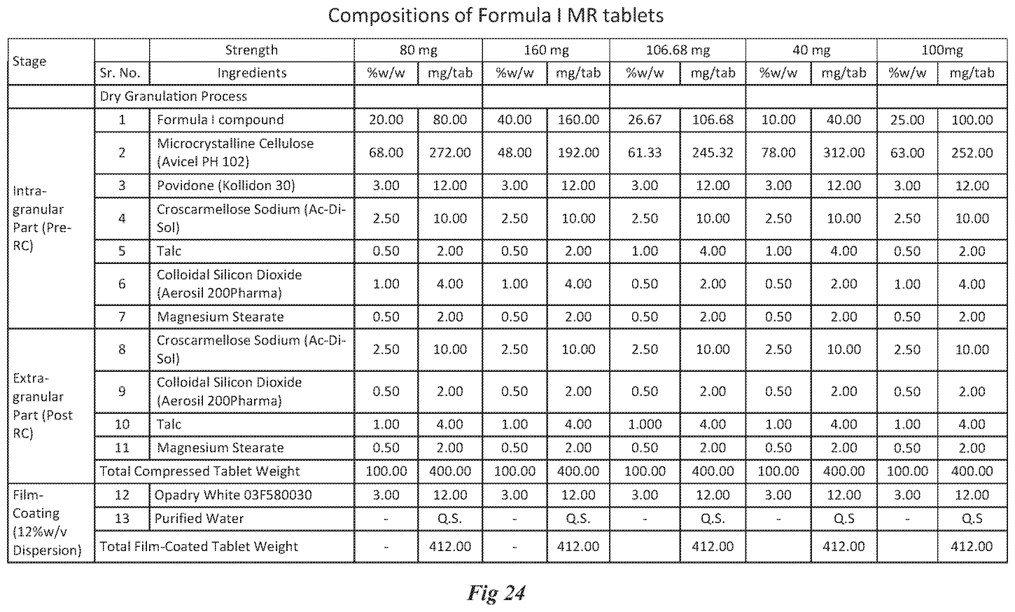

FIG. 24 shows compositions of Formula I MR tablets of 40, 80, 100, 106.68 and 160 mg.

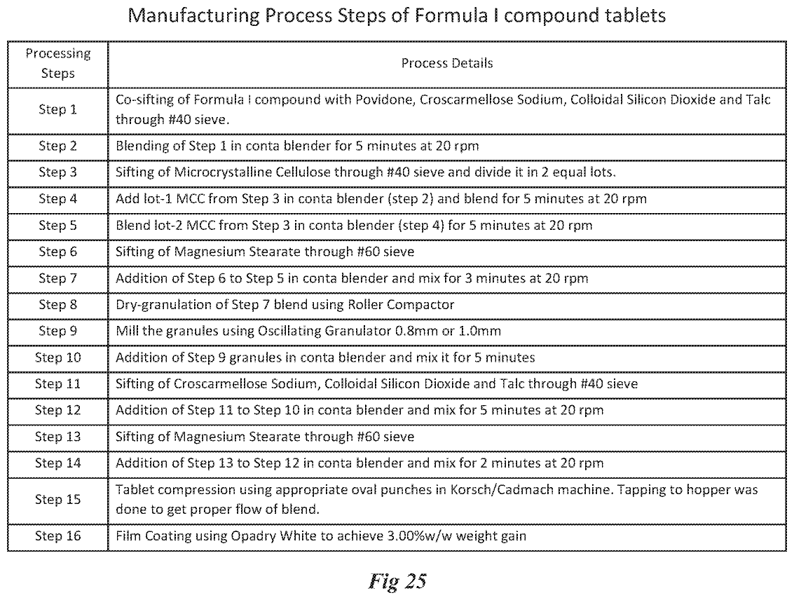

FIG. 25 shows Manufacturing Process steps to prepare Formula I compound tablets of 40, 80, 100, 106.68 and 160 mg.

### METHODS OF TREATING PARKINSON'S DISEASE AND PARKINSONISM

In another aspect, the present disclosure relates to a method of treating a disease or condition mediated, at least in part, by leucine-rich repeat kinase 2 (LRRK2) with a modified release formulation comprising a therapeutically effective amount of the Formula I compound and one or more of the excipients described herein. In particular, the disclosure provides methods for preventing or treating a disorder associated with LRRK2 in a mammal, comprising the step of administering to said mammal a therapeutically effective amount of the Formula I compound. In some embodiments, the disease or condition mediated, at least in part, by LRRK2 is a neurodegenerative disease, for example, a central nervous system (CNS) disorder, such as Parkinson's disease (PD), Parkinsonism, Alzheimer's disease (AD), dementia (including Lewy body dementia and vascular dementia), amyotrophic lateral sclerosis (ALS), age related memory dysfunction, mild cognitive impairment (e.g., including the transition from mild cognitive impairment to Alzheimer's disease), argyrophilic grain disease, lysosomal disorders (for example, Niemann-Pick Type C disease, Gaucher disease) corticobasal degeneration, progressive supranuclear palsy, inherited frontotemporal dementia and parkinsonism linked to chromosome 17 (FTDP-17), withdrawal symptoms/relapse associated with drug addiction, L- Dopa induced dyskinesia, Huntington's disease (HD), and HIV-associated dementia (HAD). In other embodiments, the disorder is an ischemic disease of organs including but not limited to brain, heart, kidney, and liver. In some embodiments, the disease is Crohn's disease.

### EXAMPLES

### Example 1

### Isolation and Physicochemical Characteristics of Formula I Compound

Formula I compound, 2-methyl-2-(3-methyl-4-(4-(methylamino)-5- (trifluoromethyl)pyrimidin-2-ylamino)-1H-pyrazol-1-yl)propanenitrile (CAS Reg. No. 1374828- 69-9), prepared according to Example 394 of US 8815882, and Compound 12 of Estrada, A. A. et al (2013) J Med. Chem. 57:921-936, each of which are specifically incorporated by reference, was dissolved in methyl tent-butylether (MTBE, 10 vol, 200 ml) to give a brown solution. This solution was filtered through 3M Zeta Plus activated carbon disc (R55SP, 5 cm diameter) at 300 ml/min. The filter was washed with MTBE (5vol, 100 ml). The clear, not colored, solution (300 ml) was concentrated to 8 vol (160 ml) and charged into a 500 ml reactor. n-Heptane (8vol, 160 ml) was added at 20° C. Solution initially remained clear but then crystallization started after 2 minutes. Temperature was increased gradually (rate 2° C/min). Full dissolution was achieved only at 69° C. Further heptane (4vol, 80 ml) was added at 70° C.; clear solution was visually observed at 70° C. The temperature was set to 65° C. (1.0° C/min). At 65° C. with the solution clear, seed crystals of the Formula I compound (200 mg, same batch) were added and they did not dissolve. The temperature was then lowered to 20° C. over the course of 8 hrs. It was stirred at 20° C. overnight. Solid was filtered and washed two times with the mother liquors. It was dried under vacuum at 40° C. for 2 hrs. to give 15.91 g of crystalline Formula I compound (79.6% yield). Mother liquors were evaporated to dryness to give additional 3.47 g (17.4% recovery).

A Form C polymorph of Formula I compound was obtained from block-like single crystals in an n-butyl acetate/cyclohexane solvent mixture system (n-butyl acetate was the solvent while cyclohexane was the anti-solvent) via liquid vapor diffusion at RT.

A Form D polymorph of Formula I compound was obtained from block-like single crystals in an acetone/n-heptane (1:10, v/v) solvent mixture system via slow evaporation at RT.

Single Crystal Structure Determination were made from the colorless block-like single crystals selected from the Form C single crystals or Form D single crystals and wrapped with Paratone-N (an oil based cryoprotectant). The crystals were mounted on a mylar loop in a random orientation and immersed in a stream of nitrogen at 150 K. Preliminary examination and data collection were performed on an Agilent SuperNova® (Cu/Kₐ λ=1.54178 Å) diffractometer and analyzed with the CrysAlisPro® (Agilent, Version:1.171.38.41) software package.

The data collection details of Form C single crystal are as follows: Cell parameters and an orientation matrix for data collection were retrieved and refined by CrysAlisPro® software using the setting angles of 6568 reflections in the range 4.0790°<θ<70.0660°. The data were collected to a maximum diffraction angle (θ) of 70.266° at 150.2(2) K. The data set was 99.9% complete having a Mean I/σ of 19.4 and D min (Cu) of 0.82 Å.

The data reduction details of Form C single crystal as follows: Frames were integrated with CrysAlisPro®, Version:1.171.38.41 software. A total of 12836 reflections were collected, of which 6205 were unique. Lorentz and polarization corrections were applied to the data. An empirical absorption correction was performed using spherical harmonics, implemented in SCALE3 ABSPACK scaling algorithm. The absorption coefficient μ of this material is 0.964 mm⁻¹ at this wavelength (λ2=1.54178 Å) and the minimum and maximum transmissions are 0.80956 and 1.00000. Intensities of equivalent reflections were averaged. The agreement factor for the averaging was 2.08% based on intensity.

The structure of Form C was solved in the space group C2/c by Direct Methods using the ShelXS™ structure solution program (Sheldrick, G. M. (2008). Acta Cryst. A64:112-122) and refined with ShelXS™, Version 2014/7 refinement package using full-matrix least-squares on F² contained in OLEX2 (Dolomanov, O. V., et al, (2009) J. Appl. Cryst. 42:339-341). All non- hydrogen atoms were refined anisotropically. The positions of hydrogen atoms occur on carbon atoms were calculated geometrically and refined using the riding model, but the hydrogen atoms occur on nitrogen atoms were refined freely according to the Fourier Maps.

The data collection details of Form D single crystal are as follows: Cell parameters and an orientation matrix for data collection were retrieved and refined by CrysAlisPro® software using the setting angles of 30349 reflections in the range 4.0180°<θ<70.5190°. The data were collected to a maximum diffraction angle (θ) of 70.562° at 150 K. The data set was 89.9% complete having a Mean I/σ of 29.3 and D min (Cu) of 0.82 Å.

The data reduction details of Form D single crystal are as follows: Frames were integrated with CrysAlisPro®, Version:1.171.38.41 software. A total of 47670 reflections were collected, of which 11179 were unique. Lorentz and polarization corrections were applied to the data. An empirical absorption correction was performed using spherical harmonics, implemented in SCALE3 ABSPACK scaling algorithm. The absorption coefficient μ of this material is 0.980 mm⁻¹ at this wavelength (λ2=1.54178 Å) and the minimum and maximum transmissions are 0.83622 and 1.00000. Intensities of equivalent reflections were averaged. The agreement factor for the averaging was 2.69% based on intensity.

The structure of Form D was solved in the space group Pca2₁ by Direct Methods using the ShelXS structure solution program and refined with ShelXS™, Version 2014/7 refinement package using full-matrix least-squares on F² contained in OLEX2. All non-hydrogen atoms were refined anisotropically. Hydrogen atom positions were calculated geometrically and refined using the riding model.

TABLE 1
Single-crystal X-ray diffraction (SCXRD) instrument parameters

| Instrument | Agilent SuperNova |
|---|---|
| X-Ray sources generator | SuperNova Microfocus X-ray Source |
| | (Cu/K_{α}: 1.54184 Å) |
| | 50 KV, 0.8 mA |
| Detector | Eos CCD detector |
| | (Detector resolution: 16.0450 pixels mm⁻¹) |
| Goniometer | Four-circle Kappa Goniometer |
| Low Temperature Devices | Oxford Cryosystems |
| Software | CrysAlisPro (Version: 1.171.38.41) |

Polymorph forms of the Formula I compound were solved using the She1XT (Sheldrick, G. M. (2015). Acta Cryst. A71, 3-8) structure solution program (Intrinsic Phasing method) and refined using SHELXL-2015 refinement package (Sheldrick, G. M. (2015). Acta Cryst. A71, 3- 8)) (full-matrix least-squares on F²) contained in OLEX2 (Dolomanov, O. V. et al, “OLEX2: a complete structure solution, refinement and analysis program”. J. Appl. Cryst. 2009, 42, 339- 341). The calculated XRPD pattern was obtained from Mercury (Macrae, C. F., et al, Appl. Cryst. (2006) 39:453-457) and the crystal structure representations were generated by Diamond. The single crystal X-ray diffraction data was collected at 296 K using Bruker D8 VENTURE diffractometer (Mo/Kα radiation, λ=0.71073 Å). Table 2 shows the crystallographic data and structure refinement of Forms C and D.

TABLE 2
Crystallographic data and structure refinement of
Formula I single crystal polymorph Forms C and D

| Parameters | Form C | Form D |
|---|---|---|
| Empirical formula | C₁₄H₁₆F₃N₇ | C₁₄H₁₆F₃N₇ |
| Formula weight | 339.34 | 339.34 |
| Temperature | 150.2(2) K | 150 K |
| Wavelength | Cu/K_{α} (λ = 1.54178 Å) | Cu/Kα (λ = 1.54178 Å) |
| Crystal system, space group | Monoclinic, C2/c | Orthorhombic, Pca2₁ |
| Unit cell dimensions | a = 13.7032(3) Å | a = 17.63410(10) Å |
| | b = 17.5697(4) Å | b = 14.03430(10) Å |
| | c = 27.4196(6) Å | c = 26.2102(2) Å |
| | α = 90° | α = 90° |
| | β = 91.982(2)° | β = 90° |
| | γ = 90° | γ = 90° |
| Volume | 6597.6(3) Å³ | 6486.56(8) Å³ |
| Z, Calculated density | 16, 1.367 g/cm³ | 16, 1.390 g/cm³ |
| Absorption coefficient | 0.964 mm⁻¹ | 0.980 mm⁻¹ |
| F(000) | 2816.0 | 2816.0 |
| Crystal size | 0.4 × 0.4 × 0.3 mm³ | 0.6 × 0.5 × 0.2 mm³ |
| 2 Theta range for data collection | 6.45° to 140.532° | 6.744° to 141.124° |
| Limiting indices | −13 ≤ h ≤ 16 | −21 ≤ h ≤ 21 |
| | −21 ≤ k ≤ 15 | −16 ≤ k ≤ 14 |
| | −31 ≤ 1 ≤ 33 | −23 ≤ 1 ≤ 31 |
| Reflections collected/ | 12836/6205 [Rᵢₙₜ = 0.0208, | 47670/11179 [Rᵢₙₜ = 0.0269, |
| Independent reflections | R_{sigma} = 0.0267] | R_{sigma} = 0.0214] |
| Completeness | 98.24% | 89.80% |
| Refinement method | Full-matrix least-squares on F² | Full-matrix least-squares on F² |
| Data/restraints/parameters | 6205/0/441 | 11179/1/881 |
| Goodness-of-fit on F² | 1.038 | 1.031 |
| Final R indices [I > 2sigma(I)] | R₁ = 0.0461, wR₂ = 0.1241 | R₁ = 0.0320, wR₂ = 0.0857 |
| Final R indexes [all data] | R₁ = 0.0518, wR₂ = 0.1281 | R₁ = 0.0339, wR₂ = 0.0872 |
| Largest diff. peak and hole | 0.85/−0.37 e.Å⁻³ | 0.19/−0.21 e.Å⁻³ |

Single crystals of Form C and Form D were prepared and analyzed by single crystal X- ray diffraction (SCXRD). The single crystal structures of Form C and Form D were determined successfully.

The SCXRD characterization confirmed that Form C crystallized in monoclinic crystal system and C2/c space group with the unit cell parameters {a=13.7032(3) Å, b=17.5697(4) Å, c=27.4196(6) Å; α=90°, β=91.982 (2)°, γ=90°}. The cell volume V was calculated to be 6597.6(3) Å³. The asymmetric unit is comprised of two molecules, indicating that Form C is an anhydrate. The calculated density of Form C is 1.367 g/cm³. The unit cell of the single crystal is comprised of sixteen molecules.

The SCXRD characterization confirmed that Form D crystallized in orthorhombic crystal system and Pca2₁ space group with unit cell parameters {a=17.63410(10) Å, =14.03430(10) Å, c=26.2102(2) Å; a=90°, β=90°, γ=90°}. The cell volume Vwas calculated to be 6486.56(8) Å³. The asymmetric unit is comprised of four molecules, indicating that Form D is an anhydrate. The calculated density of Form D is 1.390 g/cm³. The unit cell of the single crystal is comprised of sixteen molecules.

The Form C polymorph of the Formula I compound exhibits an X-ray powder diffraction pattern having characteristic peaks expressed in degrees 2-theta at approximately 6.4, 15.1, 21.2, 25.7, and 27.8. The X-ray powder diffraction pattern of Form C polymorph of the Formula I compound further comprises peaks at 16.5 and 22.1±0.05 degrees 2-theta.

The Form C polymorph of the Formula I compound exhibits an X-ray powder diffraction pattern having characteristic peaks expressed in degrees 2-theta at approximately 6.4, 8.1, 8.6, 8.8, 9.9, 10.2, 12.9, 13.8, 15.1, 15.4, 16.5, 19.8, 21.2, 22.1, 23.7, 25.7, and 27.8.

The Form C polymorph of the Formula I compound exhibits an X-ray powder diffraction pattern substantially free of peaks at 13.6 and 14.8±0.05 degrees 2-theta.

The Form D polymorph of the Formula I compound exhibits an X-ray powder diffraction pattern having characteristic peaks expressed in degrees 2-theta at approximately 9.2, 14.0, 14.8, 19.7, and 20.0.

The Form D polymorph of the Formula I compound exhibits an X-ray powder diffraction pattern having characteristic peaks expressed in degrees 2-theta at approximately 8.0, 8.7, 9.2, 9.8, 10.4, 12.9, 13.4, 14.0, 14.8, 16.4, 18.5, 19.7, 20.0, 20.8, 23.1, 23.3, 23.9, 25.5, and 25.7.

The Form D polymorph of the Formula I compound exhibits an X-ray powder diffraction pattern substantially free of peaks at 13.6±0.05 degrees 2-theta.

### Example 2

### Formulation Processes

Part 1: Manufacture of the API drug IR (Immediate Release) Pellets

An initial polymer solution is made by mixing purified water, hypromellose and polyvinyl pyrrolidone. The Formula I compound (API drug substance) is added to the polymer solution and mixed. The mixture is then sieved to produce the drug dispersion. The microcrystalline cellulose spherical seed core is charged into the fluidised bed processor bowl and the drug dispersion is sprayed onto the microcrystalline cellulose. Following loss on drying (LOD) and assay in-process controls, the resultant particles are sized to produce the API drug core pellet.

The IR coating solution is prepared by mixing purified water, hypromellose and polyethylene glycol. The API drug core pellet is charged into the fluidised bed processor bowl and the IR coating solution sprayed on to the pellets until the desired weight gain (1.5-3.0%) was achieved. Following LOD in-process control, the resultant particles are sized to produce the API drug IR Pellets which are packaged and tested.

### Part 2: Manufacture of the API Drug Modified Release Pellets (MUDS)

An initial modified release polymer solution is prepared by mixing purified water, polyethylene glycol and polyvinyl pyrrolidone. Talc is added to the solution to produce a lump free dispersion. Poly(vinyl acetate) dispersion 30% is added to the dispersion and mixed. The resultant dispersion is filtered to produce the MR coating dispersion. The API drug IR Pellets are charged into the fluidised bed and the MR coating dispersion sprayed onto the pellets until the required weight gain (3-60%) is achieved. The coated pellets are then cured for about 30 minutes to about 2 hours at a product temperature of about 40° C.-60° C. Following loss on drying in-process control, the resultant particles are sized to produce the API drug MR Pellets which are packaged and tested.

Part 3: Manufacture of the API drug MUPS (Multiple Unit Pellet System) Capsule

The required amount of API drug IR Pellets (if required) followed by the required amount of API drug MR Pellets (if required) were individually and manually weighed into each gelatin capsule. The capsule was sealed, visually assessed, weighed and packaged.

### Example 3

### Formulation of Formula I Compound in Modified Release Tablets

3.1. Modified Release Tablets with HPMC Polymers

Four tablet formulations with HPMC were prepared to control API release, the tablets containing the components in Table 3 below. Rate of release can be adjusted with addition of HPMC polymers. HPMC K100 LV results in faster release than with HPMC (Methocel K-15M CR) alone.

TABLE 3
Modified release tablets with HPMC polymers

| | Low Dose Fast | | High Dose Fast | | Low Dose Slow | | High Dose slow | |
|---|---|---|---|---|---|---|---|---|
| | Quantity | Quantity | Quantity | Quantity | Quantity | Quantity | Quantity | Quantity |
| Material | (%) | (g) | (%) | (g) | (%) | (g) | (%) | (g) |
| API | 10.00 | 15.00 | 30.00 | 45.00 | 10.00 | 15.00 | 30.00 | 45.00 |
| Mannitol 100SD | 20.00 | 30.00 | 20.00 | 30.00 | — | — | — | — |
| MCC PH102 | 45.70 | 68.55 | 25.70 | 38.55 | 65.70 | 98.55 | 45.70 | 68.55 |
| HPMC | — | — | — | — | 20.00 | 30.00 | 20.00 | 30.00 |
| (Methocel K-15M CR) | | | | | | | | |
| HPMC K100 LV | 20.00 | 30.00 | 20.00 | 30.00 | — | — | — | — |
| Povidone | 3.00 | 4.50 | 3.00 | 4.50 | 3.00 | 4.50 | 3.00 | 4.50 |
| (Kollidon 30) | | | | | | | | |
| Aerosil 200 | 0.50 | 0.75 | 0.50 | 0.75 | 0.50 | 0.75 | 0.50 | 0.75 |
| Magnesium stearate | 0.80 | 1.20 | 0.80 | 1.20 | 0.80 | 1.20 | 0.80 | 1.20 |
| Total | 100.0 | 150.00 | 100.0 | 150.00 | 100.0 | 150.00 | 100.0 | 150.00 |

Excess amount of the hydrophilic fumed silica Aerosil 200 was weighed and passed through a clean, dry 850 μM sieve and then transferred to a 1-L powder bottle and weight recorded. The bottle was placed into a Turbula mixer at 32 rpm for 1 minute. Excess amounts of MCC, API and mannitol were weighed and then passed through clean, dry 600 μM sieves. The required amounts of the MCC, API and mannitol were transferred to the powder bottle and weights recorded. The contents of the powder bottle were manually mixed using a spatula for 30 seconds, and the bottle was placed into the Turbula mixer at 32 rpm for 5 minutes. The bottle contents were sieved via a clean, dry 600 μM sieve. Excess amounts of HPMC and Povidone were weighed and then passed through clean, dry 600 μM sieves. The required amounts of the HPMC and Povidone were transferred to the powder bottle, and weight recorded. The contents of the powder bottle were manually mixed using a spatula for 30 seconds and the bottle was placed into the Turbula mixer at 32 rpm for 5 minutes. The blend was visually inspected and no lumps were seen, therefore, the blend was not sieved. Next, excess amount of magnesium stearate was passed through a clean, dry 600 μm sieve. The required amount of the sieved magnesium stearate was transferred to the powder bottle, and the weight recorded. The bottle was placed into the Turbula mixer at 32 rpm for 3 minutes and then the blend was ready for tableting. Tableting was achieved on a Natoli tablet press using an oval tooling to achieve a suitable depth of fill for all four formulations.

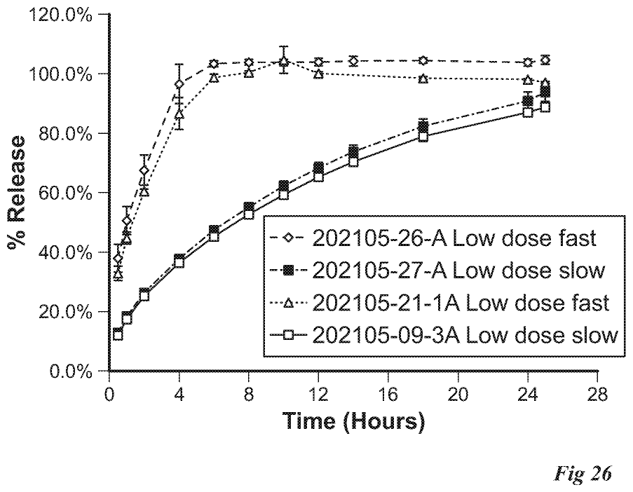

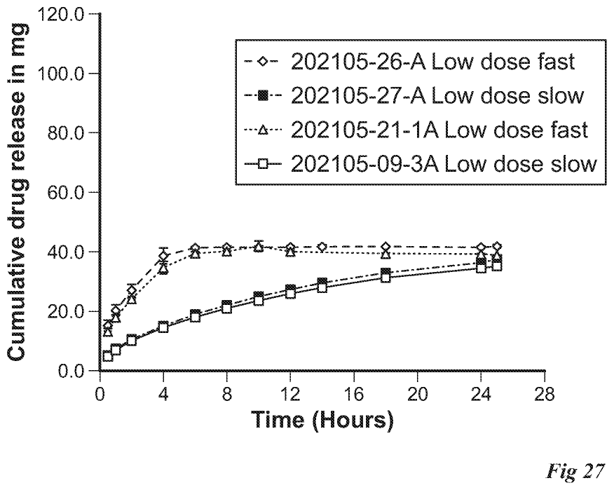

The dissolution profiles of the tablets determined using the Table 4 method are shown in FIGS. 26 and 27.

TABLE 4
Dissolution testing method for HPMC tablets

| | |
|---|---|
| Medium: | pH 3.0 McIlvaine Buffer |
| Apparatus | USP Apparatus II (Paddles) |
| Paddle Speed: | 75 rpm |
| Volume of Medium: | 900 mL |
| Temperature: | 37° C. ± 0.5° C. |
| Sinkers: | None used |
| Sampling Time Points (h): | 0.50, 1.00, 2.00, 4.00, 6.00, 8.00, 10.00, |
| | 12.00, 18.00, 24.00, 25.00 |
| Sampling Type: | Automatic (through a 10 μm free flow filter) |
| Sampling Volume: | 1.5 mL |
| Auto-sampler parameters: | Flush 3.0 mL |
| | Offset: 2.0 mL |
| | Media Recycle: On |
| | Media Replace: Off |
| | Number of Flush Times: 2 |
| | Continuous Wash: 1 |
| | Pump Flow Rates (collection, sampling, |
| | other rates): 10.0 mL/min |

3.2. Modified Release Tablets with PARTECK® Polymers

Two batches (SR PVA Matrix Tablets, 40 mg and SR PVA Matrix Tablets, 120 mg) were manufactured for the SR PVA Matrix Tablets according to Table 5.

TABLE 5

| | | SR PVA Matrix Tablets, | | | SR PVA Matrix Tablets, | | |
|---|---|---|---|---|---|---|---|
| | | 40 mg formulation | | | 120 mg formulation | | |
| | | % | | Quantity | % | | Quantity |
| Component | Function | w/w | mg/tablet | (g) | w/w | mg/tablet | (g) |
| API | Drug substance | 10.0 | 40.0 | 20.00 | 30.0 | 120.0 | 60.00 |
| Microcrystalline Cellulose | Filler | 53.0 | 212.0 | 106.00 | 33.0 | 132.0 | 66.00 |
| (Avicel PH102) | | | | | | | |
| Povidone K30 | Binder | 3.0 | 12.0 | 6.00 | 3.0 | 12.0 | 6.00 |
| Colloidal Silicon Dioxide | Glidant | 0.5 | 2.0 | 1.00 | 0.5 | 2.0 | 1.00 |
| (Aerosil 200 Pharma) | | | | | | | |
| Talc | Lubricant | 1.0 | 4.0 | 2.00 | 1.0 | 4.0 | 2.00 |
| Magnesium Stearate | Lubricant | 0.5 | 2.0 | 1.00 | 0.5 | 2.0 | 1.00 |
| Intragranular weight | — | 68.0 | 272.0 | 136.00 | 68.0 | 272.0 | 136.00 |
| Polyvinyl Alcohol | Release modifier | 30.0 | 120.0 | 60.00 | 30.0 | 120.0 | 60.00 |
| (Parteck SRP80) | | | | | | | |
| Colloidal Silicon Dioxide | Glidant | 0.5 | 2.0 | 1.00 | 0.5 | 2.0 | 1.00 |
| (Aerosil 200 Pharma) | | | | | | | |
| Talc | Lubricant | 1.0 | 4.0 | 2.00 | 1.0 | 4.0 | 2.00 |
| Magnesium Stearate | Lubricant | 0.5 | 2.0 | 1.00 | 0.5 | 2.0 | 1.00 |
| Total | | 100.0 | 400.0 | 200.0 | 100.0 | 400.0 | 200.0 |

Fifty percent of the total Microcrystalline Cellulose PH102 in addition to the API, Talc and Aerosil 200 were passed through the same 600 μm sieve and collected in a 1-L bottle. The bottle was placed into the Turbula mixer and mixed for 5 minutes at 23 rpm. The remaining 50% of Microcrystalline Cellulose PH102 in addition to Povidone K30 was passed through the same 600 μm sieve and collected in the 1-L bottle. The bottle was placed into the Turbula mixer and mixed for 5 minutes at 23 rpm. An excess amount of magnesium stearate was passed separately through clean, dry 600 μm sieves. The required amount of the magnesium stearate was added to the 1-L bottle. The bottle was placed into the Turbula mixer and mixed for 5 minutes at 23 rpm. The required amount of blend was flood filled into the die of the slug tooling (22.00 mm round flat tooling) for compression into a slug. The required compression force was applied to achieve an acceptable solid fraction (0.60 to 0.70) using the following equation (Weight/((Thickness×380.13))/1.4). Solid fraction was calculated for all slugs. The weight range targeted was 2000 mg±5%. Hardness was recorded for the first two slugs and the entire blend was slugged. The slugs were placed into a mortar and were crushed gently using a pestle into granules, taking care not to generate fine particles. The crushed slugs were passed through a 1.18 mm sieve followed by an 850 μM into a sieve receiving dish. Oversized material was returned back to mortar and pestle as needed for additional size reduction. This step was performed on 1.18 μM sieve first followed by 850 μM sieve). The retained fraction on the screen was crushed as described above until all granules pass through the 850 μM screen. The milled granules were weighed and collected into an appropriately sized amber glass bottle and the yield was found 85.98% for the 40 mg low dose formulation and 69.07% for the high dose formulation. PVA (Parteck SRP80), Silica Colloidal Anhydrous 200 (Aerosil) and talc were passed through the same 600 μm sieve and collected in the bottle. The bottle was placed into the Turbula mixer and mixed for 5 minutes at 23 rpm. Approximately 110% of magnesium stearate was passed through a 250 μm sieve and collected with weights recorded. An excess amount of magnesium stearate was passed separately through clean, dry 600 μm sieves. The required amount of the magnesium stearate was added to the 1-L bottle. The bottle was placed into the Turbula mixer and mixed for 5 minutes at 23 rpm. The required amount of tablet blend was flood filled into the die of the tablet tooling (15×7 mm oval) for compression into the first tablet. The depth of fill was adjusted to achieve the desired fill weight (400 mg±5%). The compression force was adjusted to achieve the desired hardness (12kP±2kP). The weights and thicknesses of the tablets were checked and recorded (weight range: 400mg±5%). The hardness for first two tablets was recorded. The blend was tableted to obtain around 30 tablets and hardness was collected from additional two tablets at the end of production. The acceptable tablets were packaged in a 60 mL Duma container.

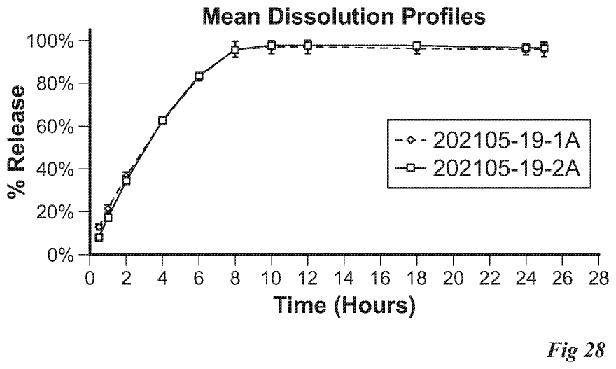

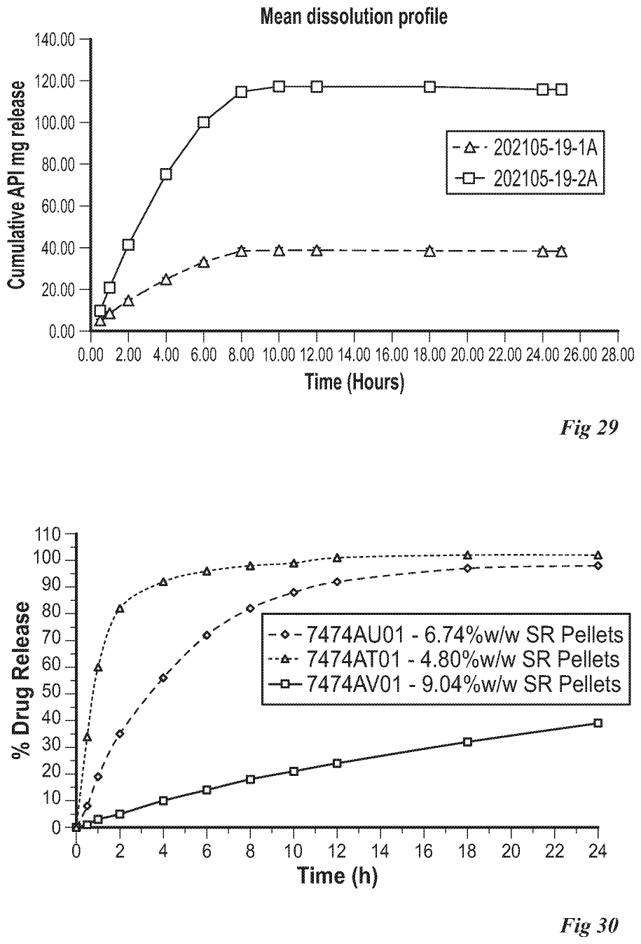

The dissolution profiles of the tablets determined according to Table 6 are shown in FIGS. 28 and 29.

TABLE 6
Dissolution Testing Method for PARTECK ® Tablets.

| | |
|---|---|
| Medium: | pH 3.0 McIlvaine Buffer |
| Apparatus | USP Apparatus II (Paddles) |
| Paddle Speed: | 75 rpm. Infinity spin at 250 rpm |
| Volume of Medium: | 900 mL |
| Temperature: | 37° C. ± 0.5° C. |
| Sinkers: | QSS2 |
| | Number of Coils: 6 (Stainless Steel) |
| | Inside Length: ~23.66 mm |
| | Inside Diameter: ~9.49 mm |
| | Max Width: ~11.79 mm |
| Sampling Time Points: | 0.50, 1.00, 2.00, 4.00, 6.00, 8.00, 10.00, |
| | 12.00, 18.00, 24.00 +1 hr at infinity spin |
| Sampling Type: | Automatic (through a 10 μm free flow filter) |
| Sampling Volume: | 1.5 mL |
| Auto-sampler parameters: | Flush 3.0 mL |
| | Offset: 2.0 mL |
| | Media Recycle: On |
| | Media Replace: Off |
| | Number of Flush Times: 2 |
| | Continuous Wash: 2 |
| | Pump Flow Rates (collection, sampling, |
| | other rates): 10.0 mL/min |

### Example 4

### Formulation of Formula I Compound in Modified Release Coated MUPS

EUDRAGIT® RS 30 D and EUDRAGIT® RL 30 D in a ratio of 9 to 1 were used as modified release polymers. The drug layer suspension was prepared by first preparing a uniform dispersion of API (250 g) and water (2.1 L) to which was then added a clear solution of PEG 6000 (8.33 g), HPMC E5 (83.33 g) and water (˜1 L). The drug layering of microcrystalline beads (CP 102, 500 g) was achieved after spraying for 10 h and 30 minutes. The drug layered product was maintained at 42° C. during the seal coating process with HPMC E5. The seal coating solution was prepared by slow addition of HPMC E5 (22.5 g) powder into water (258.8 g) with agitation until the polymer was completely dissolved. The pellets were dried for 10 minutes, then screened to retain beads between 300-425μM, and resulted in 762.3 g of the seal coated drug layered product.

The anti-tacking agent, talc (35.0 g, 50% based on dry polymer), and the plasticizer triethyl citrate (TEC) (24.0 g, 50% based on dry polymer), were added to water (312.7 g) and then homogenized for 10 minutes using a homogenizer. EUDRAGIT® RS 30 D (210.0 g) and EUDRAGIT® RL 30 D (23.3 g) were agitated for 10 minutes at low shear speed. The excipient suspension was poured slowly into the EUDRAGIT® dispersion while stirring gently with a conventional stirrer for 30 minutes. The final suspension was filtered using a 0.25 mm sieve mesh size. The suspension was kept under mixing at slow speed throughout the coating process. This process was used to prepare pellets with 5%, 10%, and 15% w/w coating. The formulations with 15% w/w coating were administered to minipigs.

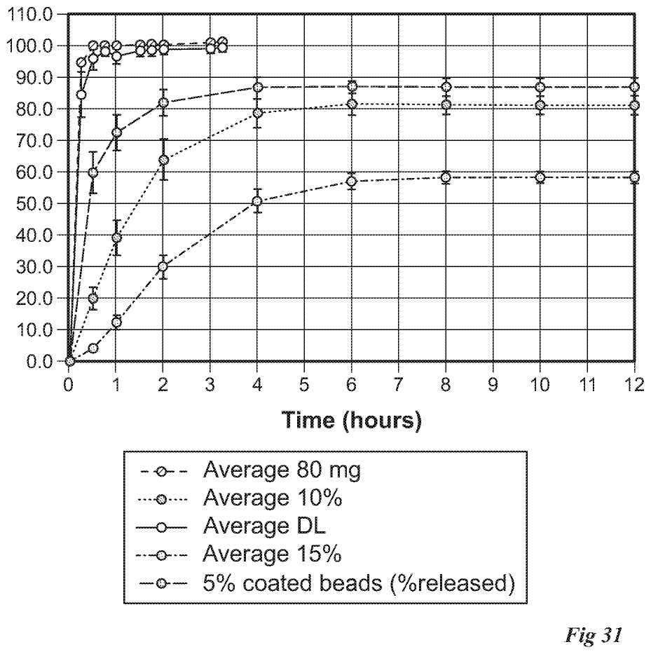

Drug release over time was next measured. The Formula I compound formulation was dissolved in Mcllvaine buffer composed of citric acid and disodium hydrogen phosphate, also known as citrate-phosphate buffer, at pH 3. Comparisons of the dissolution rates of the API (80mg), seal coated drug layered pellets, 5%, 10%, and 15% w/w coated pellets are shown in FIG. 31.

### Example 5

### Cyno PK Studies

Cynomolgus macaques with a surgically implanted CSF collection port were housed and received care according to Testing Facility IACUC Guidelines and SOPs.

Whole Blood Collection and Processing for Plasma (pharmacokinetics): Blood samples were collected from a peripheral vein via direct needle puncture at the appropriate time points (see below). Whole blood was placed onto wet ice until processed for plasma according to the Testing Facility SOP. Plasma was stored at −80° C. until shipment to the analytical laboratory on dry ice at the completion of study.

Whole Blood Collection for pharmacodynamics: Blood samples were collected from a peripheral vein via direct needle puncture at the appropriate time points. 100 μL (microliters) whole blood was pipetted into a 1.5 mL snap cap tube, snap frozen in liquid nitrogen, and stored at −80° C. until shipped to the Sponsor on dry ice at the completion of study.

CSF Collection: CSF samples were collected from an indwelling intrathecal catheter accessed via subcutaneous port using sterile technique. The port was accessed and ˜180 μL of fluid was removed from the line prior to CSF collection. CSF was quickly assessed for the presence of red blood cells, spun in a microcentrifuge at 2000g, 10 minutes, room temperature, and supernatant aliquoted, snap frozen in LN2, and stored at −80° C. until shipped to the Sponsor on dry ice at the end of the study. After CSF collection, the port/catheter was locked with ˜140 μL of sterile 0.9% sodium chloride solution.

Pharmacokinetic Study in CSF Ported Cynomolgus Monkeys: Prior to the first day of dosing, all animals (n=16) were dosed orally with vehicle (0.5% w/v Methylcellulose, 0.1% w/v Tween 80 in reverse osmosis water) once daily for 5 days. Starting on the first day of dosing, 10 animals received a once daily oral dose of the Formula I compound formulation for three or seven days, while the remaining animals continued to be dosed daily with vehicle for three days or seven days. Food was withheld from the animals overnight prior to dosing and for at least one hour after dosing (not exceeding 3 hours).

Three MUPS capsule formulations were evaluated in cynomolgus monkey (body weight approximately 5 kg). The MUPS formulations contained the Formula I compound formulated as drug layered pellets coated with KOLLICOAT® SR 30D polymer at various levels (3%, 5% and 8% w/w) designed to provide different drug release rates. In vitro dissolution results supported further characterization with in vivo PK studies. The MUPS formulations were evaluated in cynomolgus monkeys using a cross-over design with a minimum one-week washout period.

The uncoated pellet and API in capsule formulations served as comparators with immediate release rates. A single dose (2 mg/kg Formula I compound) of each formulation was administered to fasted animals (n=4) and timed blood samples were obtained over 24 hr postdose. FIG. 20A shows PK study for Formulations in Cynomolgus monkey. 1. IR pellets in capsule; 2. KOLLICOAT® 8% pellets in capsule; 3. Neat API (Formula I compound) in capsule; 4. Enteric coated pellets in capsule; 5. KOLLICOAT® 5% pellets in capsule; 6. KOLLICOAT® 3% pellets in capsule.

Following oral administration to fasted monkeys, the uncoated pellet and PIC formulations achieved similar Tₘₐₓ, Cₘₐₓ and AUC_{0-inf}. The formulations containing KOLLICOAT® SR 30D coated pellets exhibited slower absorption of Formula I compound relative to the two immediate-release formulations as shown by a longer Tₘₐₓ and reduced Cₘₐₓ. FIG. 20B shows mean concentrations-time plots of Formula I compound in monkeys (N=4) following single oral administration of Formula I compound (2 mg/kg) as immediate release and MUPS formulations. The median Tₘₐₓ for the API in capsule formulation was 1.25 hr as compared to 2.0, 1.75 and 7.5 hr for the KOLLICOAT® 3%, 5% and 8% formulations respectively, while the corresponding mean C. values were 1.14, 0.585, 0.190 and 0.0660 respectively. The relative bioavailabilities, based on AUC ratios compared to the API in capsule, of the KOLLICOAT® 3%, 5% and 8% formulations were 84%, 40% and 20%, respectively, which indicated that the lower Cₘₐₓ for the two formulations with higher polymer content was due to a combination of slower absorption rate and a decrease in the extent of absorption.

### Example 6

### Minipig PK studies

Blood collection from minipig subjects was similar to cyno subjects of Example 5.

The uncoated drug pellet, and KOLLICOAT® 5% and 8% formulations in capsules were evaluated using a crossover design in fasted Gottingen minipigs (N=3). FIG. 17A shows mean oral concentration-time plots for Formulations 1-5 in minipigs. KOLLICOAT® pellets show slower absorption rate. Enteric coated pellets achieved similar exposure to IR pellets. 1. IR pellets in capsule; 2. KOLLICOAT®8% pellets in capsule; 3. IV (0.5 mg/kg); 4. Enteric coated pellets in capsule; and 5. KOLLICOAT®5% pellets in capsule. The KOLLICOAT® 5% and 8% formulations at 1 mg/kg exhibited slower absorption of Formula I compound. Median Tmax values were 2.0, 2.5 and 4.0 hr for the uncoated pellet, and KOLLICOAT® 5% and 8% formulations, respectively, while the corresponding Cₘₐₓ values were 0.197, 0.0940 and 0.0469 μM, respectively. FIG. 17B shows mean concentrations-time plots of Formula I compound in minipigs (N=3) following single oral administration of Formula I compound (1 mg/kg) as immediate release and MUPS formulations.

### Example 7

### MUPS Pellets

MUPS pellets containing 80 mg of the compound of Formula I were prepared according to the preceding examples. Pellets with KOLLICOAT® SR 30D coatings at 5%, 7%, and 9% weight gain relative to the uncoated pellet were found to have the dissolution profiles shown in Table 7. Dissolution profile of MUPS pellets not coated with a modified release polymer is shown in Table 8. Components and drug layering steps of the 5% and 7% KOLLICOAT® SR 30D coatings are show in Tables 9 and 10.

TABLE 7
Dissolution of modified release MUPS pellets coated
with KOLLICOAT ® to different weight gains.
USPII Apparatus; 900 mL; 100 rpm; 37° C. pH 3.0 McIlvaine Buffer

% Dissolved (Mean)

| Time (h) | 5% weight gain | 7% weight gain | 9% weight gain |
|---|---|---|---|
| 0.5 | 8 | 2 | 2 |
| 1 | 19 | 5 | 4 |
| 2 | 35 | 11 | 8 |
| 4 | 56 | 22 | 14 |
| 6 | 72 | 31 | 21 |
| 8 | 82 | 39 | 27 |
| 10 | 88 | 46 | 33 |
| 12 | 92 | 52 | 38 |
| 18 | 97 | 65 | 51 |
| 24 | 98 | 76 | 64 |

TABLE 8
Dissolution of immediate release MUPS pellets

| Time (min) | % Dissolved (Mean) |
|---|---|
| 5 | 9 |
| 10 | 48 |
| 15 | 82 |
| 20 | 90 |
| 30 | 93 |
| 45 | 95 |
| 60 | 96 |
| 120 | 98 |

TABLE 9
MUPS pellets with 80 mg API and KOLLICOAT ® SR
30D coatings at 5% weight gain.

| Step | Ingredients | % w/w | mg/unit |
|---|---|---|---|
| 1 | MCC Spheres | 56.91 | 160.00 |
| 2 | API | 28.46 | 80.00 |
| 3 | Hydroxypropyl methlycellulose | 5.34 | 15.00 |
| 4 | Povidone | 2.67 | 7.50 |
| | Subtotal | 93.37 | 262.50 |

Seal coating (2% weight gain)

| | | | |
|---|---|---|---|
| 5 | Drug layered beads | 93.37 | 262.50 |
| 6 | Hydroxypropyl methlycellulose | 1.68 | 4.73 |
| 7 | Polyethylene glycol | 0.18 | 0.52 |
| | Subtotal | 95.24 | 267.75 |

MR Coating (5% weight gain)

| | | | |
|---|---|---|---|
| 8 | Seal coated beads | 95.24 | 267.75 |
| 9 | Kollicoat SR30D | 3.50 | 9.84 |
| 10 | Polyethylene glycol | 0.28 | 0.79 |
| 11 | Talc | 0.28 | 0.79 |
| 12 | Povidone | 0.70 | 1.97 |
| | Total | 100 | 281.14 |

TABLE 10
MUPS pellets with 80 mg API and KOLLICOAT ® SR
30D coatings at 7% weight gain.

| Step | Ingredients | % w/w | mg/unit |
|---|---|---|---|
| 1 | MCC Spheres | 55.85 | 160.00 |
| 2 | API | 27.92 | 80.00 |
| 3 | Hydroxypropyl methylcellulose | 5.24 | 15.00 |
| 4 | Povidone | 2.62 | 7.50 |
| | Subtotal | 91.63 | 262.50 |

Seal coating (2% weight gain)

| | | | |
|---|---|---|---|
| 5 | Drug layered beads | 91.63 | 262.50 |
| 6 | Hydroxypropyl methylcellulose | 1.65 | 4.73 |
| 7 | Polyethylene glycol | 0.18 | 0.52 |
| | Subtotal | 93.46 | 267.75 |

MR Coating (7% weight gain)

| | | | |
|---|---|---|---|
| 8 | Seal coated beads | 93.46 | 267.75 |
| 9 | Kollicoat SR30D | 4.81 | 13.78 |
| 10 | Polyethylene glycol | 0.38 | 1.10 |
| 11 | Talc | 0.38 | 1.10 |
| 12 | Povidone | 0.96 | 2.76 |
| | Total | 100 | 286.49 |

### Example 8

### Modified Release Tablets

Tablets containing 80 mg of the compound of the compound of Formula 1 (API) were prepared with PVA or HPMC release modifying agents as shown in Tables 11 and 12. Their dissolutions profiles are shown in Table 13. In comparison the dissolution profile of the API in capsule formulation (80 mg of compound of Formula I in gelatin capsule without any added excipients) is shown in Table 14.

TABLE 11
Polyvinyl alcohol based sustained release 80 mg tablets

| Ingredients | % w/w |
|---|---|
| API | 20 |
| Microcrystalline Cellulose | 43 |
| Polyvinyl pyrrolidone | 3 |
| Polyvinyl alcohol | 30 |
| Colloidal Silica, Anhydrous | 1 |
| Talc | 2 |
| Magnesium Stearate | 1 |
| Total | 100 |

TABLE 12
HPMC based sustained release 80 mg tablets

| Ingredients | % w/w |
|---|---|
| API | 20.0 |
| Mannitol | 10.0 |
| Microcrystalline Cellulose | 45.7 |
| HPMC K15M | 10 |
| HPMC K100LV | 10 |
| Povidone | 3 |
| Colloidal Silica, Anhydrous | 0.5 |
| Magnesium Stearate | 0.8 |
| Total | 100 |

TABLE 13
Dissolution Profile of HPMC and PVA Tablets

% Dissolved

| Time (h) | API 80 mg (PVA) | API 80 mg (HPMC) |
|---|---|---|
| 0.5 | 9.1 | 18.7 |
| 1 | 18.1 | 24.9 |
| 2 | 33.8 | 33.2 |
| 4 | 62.2 | 47.2 |
| 6 | 85.9 | 60.4 |
| 8 | 97.9 | 72.0 |
| 10 | 99.8 | 81.2 |
| 12 | 99.8 | 87.7 |
| 14 | 99.8 | 92.4 |
| 18 | 99.6 | 97.0 |
| 24 | 99.6 | 98.4 |

TABLE 14
Dissolution Profile of API in Capsule

| Time (min) | % Dissolved |
|---|---|
| 0 | 0 |
| 5 | 4 |
| 10 | 24 |
| 20 | 65 |
| 30 | 87 |
| 45 | 100 |

### Example 9

### Pharmacokinetic (PK) and Bioavailability Study to Investigate Modified Release Formulations in Humans

The in vivo human bioavailability of certain of the modified release (MR) formulations described in the above Examples was assessed in a healthy volunteer bioavailability and pharmacokinetic study using a cross-over design with a minimum one-week washout period. The immediate release (IR) formulation of the Formula I compound as an API-in-capsule formulation was used as a comparator and reference. A single 80 mg dose of Formula I compound was given to fasted human subjects as the API in capsule, KOLLICOAT® SR 30D 5% pellets in capsule or KOLLICOAT® SR 30D 7% pellets in capsule formulation. Timed blood samples were obtained over 72 hr postdose. The above dosing periods were repeated to acquire PK and bioavailability data for each of the above formulations as desired. Blood samples from the subjects were taken at regular intervals. PK parameters were measured using standard techniques. Additional measurements were made related to safety post-dosing, including assessment of safety laboratory tests (hematology, clinical chemistry and urinalysis), vital signs, ECGs, physical examinations and any adverse events (AEs). PK properties of the tested formulations are shown in Tables below.

TABLE 15
Pharmacokinetic Properties of MUPS
Capsules Compared to API in Capsule

| Formulation (Single | | Reduction in Cₘₐₓ vs |
|---|---|---|
| dose: 80 mg) | Observed Cₘₐₓ/C_{12 hr} Ratio | API in Capsule |
| API in capsule | 10.0 | NA |
| Kollicoat 5% MUPS | 7.77 | −39.8% |
| Kollicoat 7% MUPS | 4.39 | −69.6% |

The MUDS KOLLICOAT® SR 30D 5% and 7% pellet-in-capsule formulations exhibited reduced Cₘₐₓ and Cₘₐₓ/C₁₂ₕᵣ relative to the immediate release formulation, and were found to be were found to be well tolerated and bioavailable.

Two tablet formulations were evaluated in healthy human volunteers in a similar study design. HPMC (80 mg) and PVC (80 mg) tablets were studied in a crossover manner with timed blood samples obtained over 72 hr postdose. The API in capsule formulation (80 mg) served as the comparator. Oral administration of HPMC and PVA tablets achieved reduced Cₘₐₓ compared to fasted healthy subjects were found to be well tolerated and bioavailable.

TABLE 16
Pharmacokinetic Properties of Modified Release
Tablets Compared to API in Capsule

| Formulation (Single | | Reduction in Cₘₐₓ vs |
|---|---|---|
| dose: 80 mg) | Observed Cₘₐₓ/C_{12 hr} Ratio | API in Capsule |
| API in capsule | 8.02 | NA |
| HPMC | 2.19 | −75% |
| PVA | 1.67 | −80% |

Overall, the clinical study demonstrated that the modified release capsules and tablets were well tolerated and achieved lower Cmax and reduced Cₘₐₓ/C₁₂ₕᵣ, while maintaining oral bioavailability (relative oral bioavailability compared to API in capsule of greater than 30%). No clinically significant impact on pulse rate or blood pressure were observed. Formulations that lower Cₘₐₓ while retaining oral bioavailability may allow for higher and/or reduced dosing frequency and provide greater tolerability and safety.

Although the foregoing invention has been described in some detail by way of illustration and example for purposes of clarity of understanding, the descriptions and examples should not be construed as limiting the scope of the invention. Accordingly, all suitable modifications and equivalents may be considered to fall within the scope of the invention as defined by the claims that follow. The disclosures of all patent and scientific literature cited herein are expressly incorporated in their entirety by reference.

1. A modified release formulation comprising a therapeutically effective amount of 2-methyl-2-(3-methyl-4-(4-(methylamino)-5-(trifluoromethyl)pyrimidin-2-ylamino)- 1H-pyrazol-1-yl)propanenitrile and at least one release-modifying agent.

2. The modified release formulation of claim 1 comprising pellets containing 2-methyl-2-(3-methyl-4-(4-(methylamino)-5-(trifluoromethyl)pyrimidin-2-ylamino)- 1H-pyrazol-1-yl)propanenitrile and coated with the at least one release-modifying agent.

3. The formulation of claim 1, wherein release of 2-methyl-2-(3-methyl-4-(4-(methylamino)-5-(trifluoromethyl)pyrimidin-2- ylamino)-1H-pyrazol-1-yl)propanenitrile is less than 60% at two hours and greater than 60% at 8 hours when tested using USP Type-II Apparatus at 50-75 rpm and 37° C. in pH 3 Mcllvaine buffer, wherein the formulation is a tablet.

4. The formulation of claim 2, wherein release of 2-methyl-2-(3-methyl- 4-(4-(methylamino)-5-(trifluoromethyl)pyrimidin-2-ylamino)-1H-pyrazol-1-yl)propanenitrile is less than 60% at one hour when tested using USP Type-II Apparatus at 100 rpm and 37° C. in pH 3 Mcllvaine buffer, wherein the formulation is a capsule containing pellets.

5. The formulation of claim 1, wherein the 2-methyl-2-(3-methyl-4-(4-(methylamino)-5-(trifluoromethyl)pyrimidin-2- ylamino)-1H-pyrazol-1-yl)propanenitrile has a Cₘₐₓ, that is decreased relative to an immediate release formulation after administration to a subject.

6. The formulation of claim 5, wherein the Cₘₐₓ is decreased by at least 20%.

7. The formulation of claim 1, wherein the modified release formulation comprises 10% to 50% by weight of 2-methyl-2-(3- methyl-4-(4-(methylamino)-5-(trifluoromethyl)pyrimidin-2-ylamino)-1H-pyrazol-1- yl)propanenitrile.

8. The formulation of claim 1, wherein the 2-methyl-2-(3-methyl-4-(4-(methylamino)-5-(trifluoromethyl)pyrimidin-2- ylamino)-1H-pyrazol-1-yl)propanenitrile is crystalline.

9. The method of claim 8, wherein crystalline 2-methyl-2-(3-methyl-4-(4- (methylamino)-5-(trifluoromethyl)pyrimidin-2-ylamino)-1H-pyrazol-1-yl)propanenitrile is milled or micronized.

10. The formulation of claim 1, wherein the release-modifying agent comprises from 3% to 60% by weight of the formulation.

11. The formulation of claim 1, wherein the release-modifying agent is selected from the group consisting of MCC (Microcrystalline cellulose), HPC (Hydroxypropyl cellulose), HPMC (Hydroxypropyl methylcellulose), PEG (Polyethylene glycol glycerides), PVA (Polyvinyl alcohol), PVP (Polyvinylpyrrolidone), CAP (Cellulose acetate phthalate), CMC-Na (Carboxymethylcellulosesodium), HPMCAS (Hydroxypropyl methylcellulose acetate succinate), HPMCP (Hydroxypropyl methylcellulose phthalate), Poly(methylacrylate-co-methyl methacrylate-co-methacrylic acid), Poly(methacrylic acid-co-ethyl acrylate), Poly(methacrylic acid-co-methyl methacrylate), CA (Cellulose acetate); CAB (Cellulose acetate butyrate); EC (Ethylcellulose), Poly(ethyl acrylate-co-methyl methacrylate), Poly(ethyl acrylate-co-methyl methacrylate-co-trimethylammonium ethyl methacrylate chloride), PVAc (Polyvinyl acetate), and HPMC/CMC.

12. The formulation of claim 1, wherein the release-modifying agent is selected from the group consisting of Aquacoat®, Walocel®, HP 50/HP 55, Aqoat®, EUDRAGIT® FS 30 D, EUDRAGIT® L 30 D-55/L 100-55, EUDRAGIT® L 12,5/EUDRAGIT® L 100, EUDRAGIT® S 12,5/EUDRAGIT® S 100, Carbopol® polymers, Eastman CA, Eastman CAB, Eastman CAB, Ethocel™, Aquacoat® ECD, or Surelease®, or Glyceride GatteCoat™, EUDRAGIT® NE 30 D, EUDRAGIT® NM 30 D, EUDRAGIT® RL 30 D, EUDRAGIT® RL 100/RL PO, EUDRAGIT® RS 30 D, EUDRAGIT® RS 100/RS, Kollicoat® SR 30 D, Walocel® HM-PPA, Kollicoat® MAE 30 DP/100 P, and Eastacryl 30 D.

13. The formulation of claim 1, wherein the release-modifying agent is selected from the group consisting of microcrystalline cellulose, hydroxypropyl methylcellulose, polyethylene glycol, polyvinyl alcohol, polyvinyl acetate, polyvinylpyrrolidone, KOLLICOAT®, CARBOPOL®, and AQUACOAT®.

14. The formulation of claim 1, comprising one or more excipients selected from the group consisting of microcrystalline cellulose, hydroxypropyl methylcellulose, croscarmellose sodium, polyethylene glycol, polyvinyl alcohol, polyvinyl acetate, polyvinylpyrrolidone, purified talc, colloidal silicon dioxide, and magnesium stearate, and a coating.

15. The formulation of claim 1, wherein the formulation is a tablet.

16. The formulation of claim 15, wherein the tablet comprises 10 to 500 mg of 2-methyl-2-(3-methyl-4-(4-(methylamino)-5-(trifluoromethyl)pyrimidin-2-ylamino)-1H- pyrazol-1-yl)propanenitrile.

17. The formulation of claim 15, wherein the tablet comprises 40 to 120 mg of 2-methyl-2-(3-methyl-4-(4-(methylamino)-5-(trifluoromethyl)pyrimidin-2-ylamino)-1H- pyrazol-1-yl)propanenitrile.

18. The formulation of claim 15, wherein the tablet comprises 30 to 80 mg of 2-methyl-2-(3-methyl-4-(4-(methylamino)-5-(trifluoromethyl)pyrimidin-2-ylamino)-1H- pyrazol-1-yl)propanenitrile.

19. The formulation of claim 15, wherein the release-modifying agent is HPMC.

20. The formulation of claim 15, wherein the release-modifying agent is a PARTECK® polymer.

21. The formulation of claim 19 wherein the release-modifying agent comprises 20-30% w/w of the formulation.

22. The formulation of claim 1, wherein the formulation is a capsule containing pellets.

23. The formulation of claim 22, wherein the capsule is a multi-unit particulate combination of immediate release pellets and modified release pellets contained in the capsule.

24. The formulation of claim 22, wherein the pellets comprise a release-modifying agent selected from KOLLICOAT®, CARBOPOL®, and AQUACOAT®.

25. The formulation of claim 22, wherein the formulation is a multi-unit particulate combination of immediate release pellets and delayed release pellets contained in a capsule.

26. The formulation of claim 22, wherein the modified release formulation is selected from a delayed-release pellet formulation, a controlled- release pellet formulation, an extended-release pellet formulation, and a pulsatile-release pellet formulation.

27. The formulation of claim 22, wherein the formulation comprises a coating agent, wherein the coating agent is EUDRAGIT®.

28. The formulation of claim 27, wherein the coating agent comprises from 3% to 60% of EUDRAGIT® by weight of the formulation.

29. The formulation of claim 28, wherein the coating agent comprises EUDRAGIT® RS 30 D of up to 20% w/w.

30. The formulation of claim 29, wherein the coating agent comprises EUDRAGIT® NM 30 D of up to 60% w/w.

31. The formulation of claim 22, wherein the formulation comprises a coating agent, wherein the coating agent is KOLLICOAT® SR 30D.

32. The formulation of claim 31 wherein the KOLLICOAT® SR 30D provides about a 5% weight gain.

33. The formulation of claim 31 wherein the KOLLICOAT® SR 30D provides about a 7% weight gain.

34. The formulation of claim 31 wherein the KOLLICOAT® SR 30D provides about a 8% weight gain.

35. The formulation of claim 31 wherein the KOLLICOAT® SR 30D provides a 5-9% weight gain.

36. A method of preparing a modified release formulation comprising:
(a) coating an inert core selected from the group consisting of sugar, MCC and tartaric acid, with 2-methyl-2-(3-methyl-4-(4-(methylamino)-5 -(trifluoromethyl)pyrimidin-2- ylamino)-1H-pyrazol-1-yl)propanenitrile to form an API-core pellet;
(b) coating the API-core pellet with a cosmetic, non-functional seal coating to form a seal-coated pellet; and
(c) coating the seal-coated pellet with a release-modifying agent to form the modified release formulation.

37. The method of claim 36 wherein the inert core is selected from a sugar, microcrystalline cellulose (MCC), tartaric acid, polyols, carnauba wax, silicon dioxide, and combinations thereof.

38. The method of claim 36 wherein the cosmetic, non-functional seal coating is selected from hydroxypropyl methylcellulose (UPMC), and a mixture of hypromellose and ethylcellulose.

39. The method of claim 38 wherein the release-modifying agent is selected from the group consisting of KOLLICOAT®, EUDRAGIT®, hydroxypropyl methylcellulose (HPMC), and a mixture of hypromellose and ethylcellulose.

40. A method of preparing a modified release formulation comprising:
(a) roller compaction of 2-methyl-2-(3-methyl-4-(4-(methylamino)-5- (trifluoromethyl)pyrimidin-2-ylamino)-1H-pyrazol-1-yl)propanenitrile and one or more excipients selected from the group consisting of microcrystalline cellulose, hydroxypropyl methylcellulose, croscarmellose sodium, polyethylene glycol, polyvinyl alcohol, polyvinyl acetate, polyvinylpyrrolidone, purified talc, colloidal silicon dioxide, and magnesium stearate, whereby a pellet is formed; and
(b) polymer coating the pellet with a dispersion of a coating agent selected from KOLLICOAT®, CARBOPOL®, AQUACOAT®, and OPADRY® White.

41. The method of claim 40 further comprising one or more steps selected from extrusion, spheronization, and compression.

42. The method of claim 40 further comprising filling a soft or hard capsule shell with the coated pellets.

43. A method of preparing a modified release formulation tablet comprising:
(a) blending a dry mixture of 2-methyl-2-(3-methyl-4-(4-(methylamino)-5- (trifluoromethyl)pyrimidin-2-ylamino)-1H-pyrazol-1-yl)propanenitrile, povidone, croscarmellose sodium, silicon dioxide, talc, microcrystalline cellulose, and magnesium stearate;
(b) preparing a dry-granulation of the dry mixture by roller compaction as granules;
(c) milling the granules;
(d) adding croscarmellose sodium, silicon dioxide, talc, and magnesium stearate to the milled granules to form an extra-granular mixture;
(e) compressing the extra-granular mixture into tablets; and
(f) coating the tablets with a coating agent selected from KOLLICOAT®, CARBOPOL®, AQUACOAT®, and EUDRAGIT®.

44. A method of treating a LRRK2 mediated disease comprising administering to a subject in need thereof a formulation of claim 1.

45. The method of claim 44 wherein one or more of the formulations are administered to the subject once per day, twice per day, or three times per day.

46. The method of claim 45 wherein the formulations are administered to the subject twice per day.

47. The method of claim 46 wherein the LRRK2 mediated disease is a neurodegenerative disease.

48. The method of claim 47 wherein the LRRK2 mediated disease is Parkinson's disease.
